(19) United States
(12) Patent Application Publication
(10) Pub. No.: US 2025/0244429 A1
(43) Pub. Date: Jul. 31, 2025

(54) IMAGE PROCESSING DEVICE, OPERATION METHOD OF IMAGE PROCESSING DEVICE, AND OPERATION PROGRAM OF IMAGE PROCESSING DEVICE

(71) Applicant: FUJIFILM Corporation, Tokyo (JP)

(72) Inventor: Miyuki TAKAHASHI, Tokyo (JP)

(73) Assignee: FUJIFILM Corporation, Tokyo (JP)

(21) Appl. No.: 19/026,136

(22) Filed: Jan. 16, 2025

(30) Foreign Application Priority Data
Jan. 26, 2024 (JP) 2024-010461

(51) Int. Cl. G01R 33/56; G06T 11/00

(52) U.S. Cl. CPC G01R 33/5608; G06T 11/003; G06T 2211/40

(57) ABSTRACT

An image processing device includes a processor that performs image processing on a plurality of slice images which are output from a tomography apparatus and which represent an axial cross section orthogonal to a body axis of a subject, in which the processor acquires the plurality of slice images, and generates a tomographic image representing a designated cross section by using a function having pixel values of the plurality of slice images as parameters.

### CROSS-REFERENCE TO RELATED APPLICATIONS

The present application claims priority under 35 U.S.C. § 119 to Japanese Patent Application No. 2024-010461 filed on Jan. 26, 2024. The above application is hereby expressly incorporated by reference, in its entirety, into the present application.

### BACKGROUND

### 1. Technical Field

The technology of the present disclosure relates to an image processing device, an operation method of an image processing device, and an operation program of an image processing device.

### 2. Description of the Related Art

A tomography apparatus that captures a tomographic image of a subject, such as a computed tomography (CT) apparatus and a magnetic resonance imaging (MRI) apparatus, is known. The tomography apparatus outputs a plurality of slice images representing an axial cross section orthogonal to a body axis of the subject. In addition, the tomographic image representing any cross section of the subject is also acquired by generating an isotropic three-dimensional image in which three-dimensional resolution is made isotropic based on the plurality of slice images captured by the tomography apparatus and cutting out a tomographic image representing any cross section from the isotropic three-dimensional image. Such a tomographic image representing the cross section is called a multi planar reconstruction (MPR) image or the like.

In a tomography apparatus disclosed in JP2005-143735A, three-dimensional preparation image data (corresponding to a three-dimensional image) is generated from slice images, and a tomographic image is acquired by designating any cross section on the generated three-dimensional preparation image data.

### SUMMARY

However, a process of generating the three-dimensional image from the slice images has a large load of image processing and takes a long time, and thus it takes a long time to display the tomographic image of the designated cross section. Moreover, in a case in which such three-dimensional image data is generated, for example, based on all the slice images of an imaging range set in a body axis direction, the three-dimensional image data is generated after the imaging of the tomography apparatus is completed. In this case, it takes longer time. Therefore, there has been a demand for shortening a time from the start of the imaging via the tomography apparatus to the display of the tomographic image of the designated cross section.

The technology according to the present disclosure provides an image processing device, an operation method of an image processing device, and an operation program of an image processing device capable of displaying a tomographic image of a designated cross section in a shorter time than in the related art.

The technology of the present disclosure provides an image processing device comprising a processor that performs image processing on a plurality of slice images which are output from a tomography apparatus and which represent an axial cross section orthogonal to a body axis of a subject, in which the processor acquires the plurality of slice images, and generates a tomographic image representing a designated cross section by using a function having pixel values of the plurality of slice images as parameters.

It is preferable that, in a case in which the tomography apparatus outputs the plurality of slice images during imaging, the processor starts processing of generating the tomographic image during the imaging by using the plurality of slice images.

It is preferable that the function is created in accordance with an imaging condition of the tomography apparatus, the imaging condition including at least a slice interval of the slice images.

It is preferable that the processor acquires the imaging condition to create the function before the tomography apparatus starts the imaging.

It is preferable that, in a case in which a body axis direction of the subject is a Z axis direction and two directions defining a cross section orthogonal to the body axis direction are an X axis direction and a Y axis direction, the function includes an isotropic data derivation function for deriving isotropic data as a pixel value of a pixel in which a resolution is made isotropic in the X axis direction, the Y axis direction, and the Z axis direction, and the processor generates the tomographic image based on the isotropic data.

It is preferable that the cross section of the tomographic image is any one of an axial cross section, a sagittal cross section, or a coronal cross section.

It is preferable that the processor receives designation of a slab thickness as a thickness of the tomographic image, and generates the tomographic image by performing weighting in accordance with the slab thickness.

It is preferable that, in a case in which a body axis direction of the subject is a Z axis direction, a width direction of a cross section orthogonal to the body axis direction is an X axis direction, and a height direction is a Y axis direction, the function includes a pixel value derivation function for deriving a pixel value of a tilt cross section tilted by rotating the axial cross section of the slice image about an X axis, and the processor generates a tilt image that is the tomographic image of the tilt cross section by using the pixel value derivation function.

It is preferable that, in a case in which a plurality of the tilt images arranged in the Z axis direction are generated, heights of pixels of the plurality of tilt images in the Y axis direction are aligned.

It is preferable that the processor receives designation of a slab thickness as a thickness of the tilt image, and generates the tilt image by performing weighting in accordance with the designated slab thickness.

The technology of the present disclosure provides an operation method of an image processing device including a processor that performs image processing on a plurality of slice images which are output from a tomography apparatus and which represent an axial cross section orthogonal to a body axis of a subject, the operation method comprising: via the processor, acquiring the plurality of slice images; and generating a tomographic image representing a designated cross section by using a function having pixel values of the plurality of slice images as parameters.

The technology of the present disclosure provides an operation program of an image processing device including a processor that performs image processing on a plurality of slice images which are output from a tomography apparatus and which represent an axial cross section orthogonal to a body axis of a subject, the operation program causing the processor to execute a process comprising: acquiring the plurality of slice images; and generating a tomographic image representing a designated cross section by using a function having pixel values of the plurality of slice images as parameters.

According to the technology of the present disclosure, the tomographic image of the designated cross section can be displayed in a shorter time than in the related art.

### BRIEF DESCRIPTION OF THE DRAWINGS

FIG. 1 is a diagram showing a CT apparatus.

FIG. 2 is a diagram showing a schematic configuration of the CT apparatus.

FIG. 3 is a diagram showing processing of a processor according to a first embodiment.

FIG. 4 is a diagram showing MPR image generation processing using an isotropic three-dimensional image VD_ISO in the related art.

FIG. 5 is a diagram showing a procedure of processing of creating an MPR image generation function.

FIG. 6 is a diagram conceptually showing processing of creating a Z coordinate table.

FIGS. 7A and 7B are diagrams showing a relationship between a size of an FOV and an isotropic Z coordinate.

FIG. 8 is a diagram conceptually showing processing of creating an isotropic data derivation function.

FIG. 9 is a diagram conceptually showing processing of creating a pixel value derivation function of an MPR image of an axial cross section.

FIG. 10 is a diagram conceptually showing processing of creating a pixel value derivation function of an MPR image of a sagittal cross section.

FIG. 11 is a diagram conceptually showing processing of creating a pixel value derivation function of an MPR image of a coronal cross section.

FIG. 12 is a flowchart showing a processing procedure of the MPR image generation processing.

FIG. 13 is a diagram showing processing of a processor according to a second embodiment.

FIG. 14 is a diagram showing a procedure of processing of creating an MPR image generation function.

FIG. 15 is a diagram conceptually showing processing of creating a coordinate transformation table.

FIG. 16 is a diagram showing a state in which heights of pixels of a tilt image in a Y axis direction are not aligned.

FIG. 17 is a diagram conceptually showing processing of creating a pixel value derivation function of the tilt image.

FIG. 18 is a diagram conceptually showing processing of creating a weight correction function.

FIG. 19 is a flowchart showing a processing procedure of tilt image generation processing.

FIG. 20 is a diagram showing an example in which an image display device functions as an image processing device.

### DETAILED DESCRIPTION

### First Embodiment

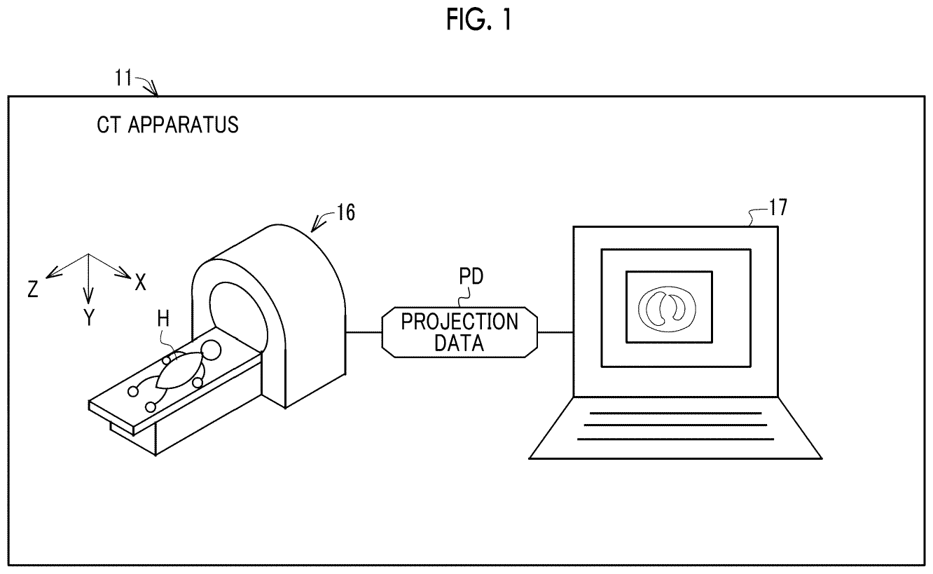

A CT apparatus 11 shown in FIG. 1 is an example of a tomography apparatus according to the technology of the present disclosure. As is well known, the CT apparatus 11 obtains a tomographic image of a subject H, which is an example of a subject, by imaging the subject H using radiation (for example, X-rays). The CT apparatus 11 is installed in, for example, an imaging room of a radiology department in a medical facility. The CT apparatus 11 comprises a stand 16 and a console 17. The console 17 functions as an operation terminal and a control device for operating the stand 16. The console 17 is operated by an operator such as a radiology technician. In addition, the console 17 also functions as an image processing device that performs image processing on data output from the stand 16 to generate the tomographic image. The console 17 is an example of an “image processing device” according to the technology of the present disclosure. The console 17 also functions as an image display device that displays the generated tomographic image.

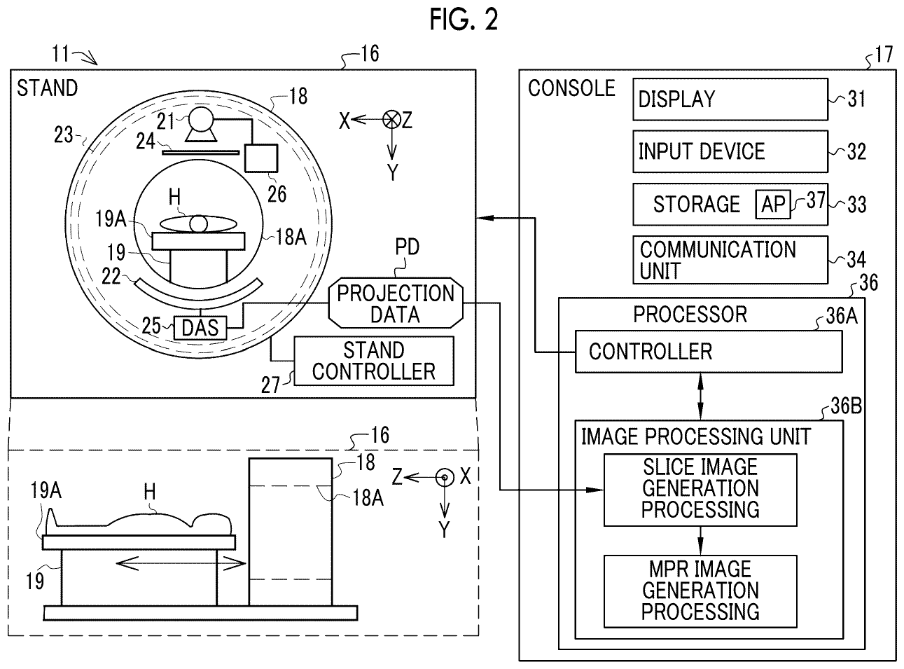

As shown in FIG. 2, the stand 16 is a main part of the CT apparatus 11, and comprises a gantry 18 and an examination table device 19. In FIG. 2, in addition to a front view of the stand 16, a side view of the stand 16 is shown in a rectangular broken line frame. The examination table device 19 has a top plate 19A on which the subject H can be placed in a decubitus posture. The subject H is placed in a posture in which a body axis matches a longitudinal direction of the top plate 19A (Z axis direction of the stand 16). The top plate 19A can be moved in the Z axis direction. The gantry 18 has an annular shape as a whole, and a circular opening part 18A having a diameter larger than a width of the top plate 19A is formed in the center. In a case of the imaging, the top plate 19A on which the subject H is placed is moved in the Z axis direction with respect to the gantry 18 to enter the opening part 18A. The imaging is performed while the top plate 19A is moved with respect to the gantry 18.

Inside the gantry 18, a radiation source 21, a detector 22, and a frame 23 are disposed. The radiation source 21 emits the radiation toward the subject H. The detector 22 is a radiation detector that detects the radiation transmitted through the subject H. The radiation transmitted through the subject H is attenuated by interaction (absorption, scattering, and the like of the radiation) with structures, such as organs and bones, in the body of the subject H. The structures each have an attenuation coefficient for the radiation peculiar to the structures, and the radiation transmitted through the structures carries intensity information reflecting the attenuation coefficient of the structures. The detector 22 has a detection surface in which pixels are two-dimensionally arranged, and outputs a detection signal in accordance with the intensity information of the radiation for each pixel in the detection surface. In addition, the detector 22 has a substantially arc shape in accordance with a curvature of the gantry 18, and the detection surface is also curved.

The radiation source 21 and the detector 22 are disposed at positions facing each other in the gantry 18 and are rotated about the Z axis while maintaining the facing posture. The frame 23 has an annular shape and supports the radiation source 21 and the detector 22 to be rotatable. In a case of imaging, the stand 16 detects projection data PD at a plurality of positions in a circumferential direction about the Z axis corresponding to the body axis of the subject H while rotating the radiation source 21 and the detector 22 about the subject H on the top plate 19A, by using the detector 22. In a case of the imaging, the top plate 19A is also moved in the Z axis direction in synchronization with the rotation of the radiation source 21 and the detector 22. As a result, the projection data PD of the radiation at each position about the body axis of the subject H is acquired.

In the stand 16, a Y axis represents a height direction, and an X axis represents a width direction. As an example, in the examination table device 19, the subject H is placed in a decubitus posture with a head side facing the gantry 18. Therefore, in the imaging, the subject H enters the gantry 18 from the head side, and the projection data PD is output in the order from the head side to a foot side.

A data acquisition system (DAS) 25 collects the detection signals output by the detector 22, generates the projection data PD at each position about the Z axis based on the collected detection signals, and outputs the generated projection data PD to the console 17.

An irradiation field limiter 24 (also referred to as a collimator) that limits an irradiation field of the radiation is disposed in front of the radiation source 21 in an irradiation direction. The irradiation field limiter 24 has an irradiation opening of which a contour is defined by a plurality of shielding plates that shield the radiation, and a size of the irradiation opening can be changed by moving the shielding plates. Reference numeral 26 denotes a high-voltage generator that generates a high voltage to be supplied to the radiation source 21. The radiation source 21 and the detector 22 are electrically connected to the frame 23 by a slip ring method, and, for example, power supply, transmission and reception of data, and the like are performed via the slip ring. The radiation source 21 and the detector 22 can perform imaging of a helical scan by the connection of the slip ring method. The helical scan method refers to a method of performing the imaging while rotating the radiation source 21 and the detector 22 in one direction without reversing the rotation direction.

A stand controller 27 is provided in the stand 16. The stand controller 27 controls each part of the stand 16 in addition to the rotation of the radiation source 21 and the detector 22 and the movement of the top plate 19A based on an instruction from the console 17.

Imaging conditions of the CT apparatus 11 are set through the stand controller 27 via the operation from the console 17. The imaging conditions include an imaging range, a slice interval, and the like in addition to an irradiation condition of the radiation of the radiation source 21. The irradiation condition of the radiation includes a tube voltage (unit: kv) applied to the radiation source 21, a tube current (unit: mA), and an irradiation time (unit: msec) of the radiation. The product of the tube current and the irradiation time defines a total irradiation amount of the radiation and is called a mAs value. The irradiation field is adjusted by, for example, changing the size of the irradiation opening of the irradiation field limiter 24 in an X-Z plane. It should be noted that, in a case in which the radiation emitted from the radiation source 21 is a conical cone beam, a width of the irradiation field in the Z axis direction can be adjusted by adjusting a width of the irradiation opening of the irradiation field limiter 24 in the Z axis direction. In addition, the imaging range of the subject H in the Z axis direction, such as an entire body or a chest to an abdomen, is adjusted by changing a movement range of the top plate 19A.

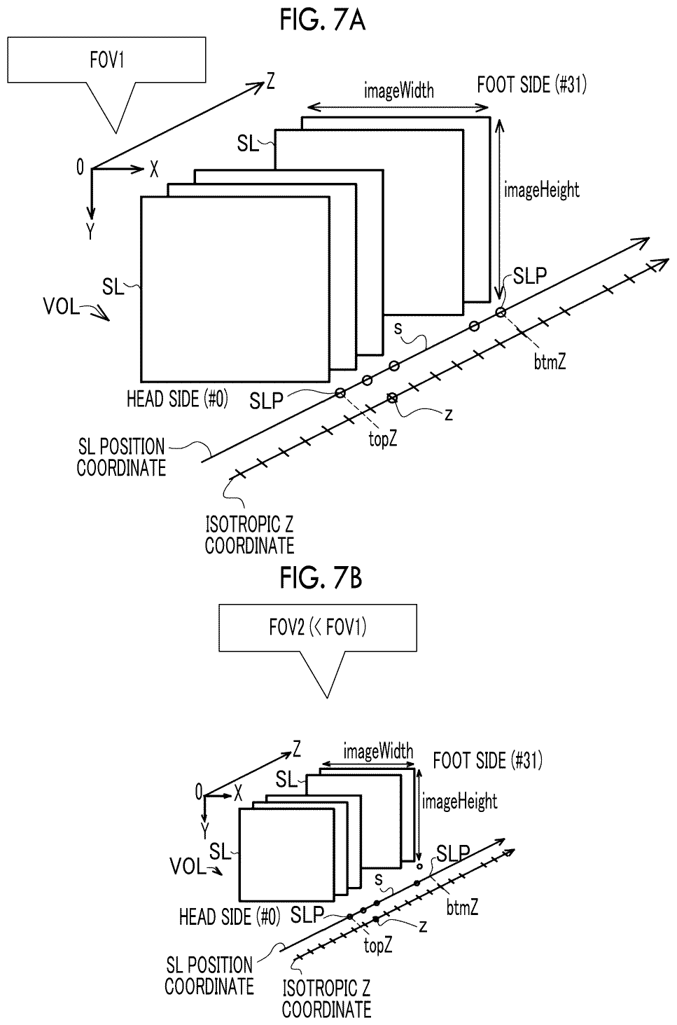

Further, it is also possible to change an imaging range of a slice image SL (refer to FIGS. 7A and 7B and the like) described later. The slice image SL is a tomographic image representing an axial cross section orthogonal to an X-Y plane, that is, the body axis of the subject H, and is generated by image reconstruction based on the projection data PD as described later. The imaging range of such a slice image SL in the X-Y plane is also referred to as a field of view (FOV). For example, in the axial cross section of the chest of the subject H, a size of the FOV can be changed such that a relatively large region in which the entire chest is included is set as the FOV or such that a relatively small region such as a part of the chest is set as the FOV. A flux of the radiation spreads toward the detector 22 with the focus of the radiation source 21 as a base point. Therefore, in a case in which an interval between the radiation source 21 and the detector 22 in the gantry 18 is constant, a region of the flux transmitted through the subject His narrowed in a case in which the subject His brought close to the radiation source 21 by adjusting a height of the top plate 19A. Since a region of the body of the subject H through which the radiation is transmitted can be imaged, the FOV is small in a case in which the region through which the radiation is transmitted is narrow. On the contrary, in a case in which the subject His moved away from the radiation source 21, the region of the flux transmitted through the subject H is widened, and thus the FOV can be increased. As described above, the size of the FOV is adjusted by, for example, adjusting the height of the top plate 19A. On the other hand, since the number of pixels of the detector 22 is determined, a resolution of the slice image SL to be captured is increased in a case in which the FOV is reduced, and the resolution is decreased in a case in which the FOV is increased. In addition, it is also possible to adjust a distance that the top plate 19A advances in the Z axis direction while the radiation source 21 and the detector 22 rotate once, that is, a movement speed in the Z axis direction. By adjusting the movement speed in the Z axis direction, the slice interval of the slice images SL can be adjusted. The FOV and the slice interval are also set as the imaging conditions.

The console 17 comprises a display 31, an input device 32, a storage 33, a communication unit 34, and a processor 36. The console 17 is configured based on, for example, a personal computer, and a hardware configuration thereof is the same as a hardware configuration of a general computer. The display 31 is, for example, a liquid crystal display, and displays an operation screen, the captured slice image SL, and the like. The input device 32 is a device for the operator to input an operation instruction, and is configured by a keyboard, a mouse, and the like.

The storage 33 is a data storage that stores various programs such as a control program for controlling the respective units of the console 17. The various programs include an application program 37 for causing the processor 36 to function as a control device and an image processing device of the CT apparatus 11. Examples of the storage 33 include a hard disk drive (HDD) and a solid-state drive (SSD). The projection data PD acquired from the stand 16 and the generated slice image SL are also temporarily stored in the storage 33. The application program 37 is an example of an “operation program” according to the technology of the present disclosure.

The communication unit 34 is a communication interface for performing communication between the console 17 and an external device such as an image database (DB) outside the stand 16. The communication unit 34 is connected to a network (not shown), such as a local area network (LAN) and/or a wide area network (WAN), and performs transmission control in accordance with a communication protocol defined in various types of wired or wireless communication standards.

The processor 36 functions as a controller 36A that controls the respective units of the console 17 and an image processing unit 36B that executes various types of image processing.

As an example, the processor 36 is configured by a central processing unit (CPU) and a memory, such as a random access memory (RAM). The CPU functions as the processor 36 by loading the various programs including the application program 37 from the storage 33 into the memory and executing the loaded programs. The processor 36 is an example of a “processor” according to the technology of the present disclosure. In addition, the console 17 including the processor 36 that executes the image processing is an example of an “image processing device” according to the technology of the present disclosure.

The controller 36A controls the stand 16 through the stand controller 27 in accordance with an instruction of the operator input from the input device 32. The setting of the imaging conditions and the like are performed by the controller 36A. The image processing unit 36B executes slice image generation processing and MPR image generation processing.

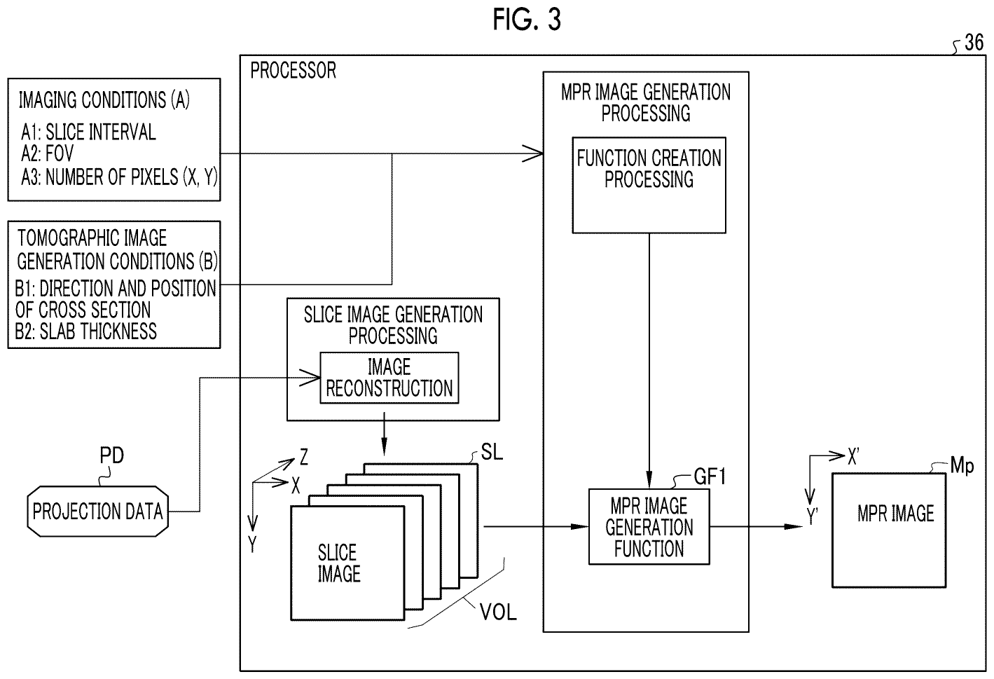

FIG. 3 shows an outline of processing of the processor 36 that functions as the image processing unit 36B. The slice image generation processing is processing of generating the tomographic image by performing the image reconstruction based on the projection data PD acquired from the stand 16. In the slice image generation processing, the reconstructed tomographic image is the tomographic image representing the axial cross section orthogonal to the body axis (Z axis) of the subject H. Here, the tomographic image is referred to as the slice image SL. The slice image SL is an example of a “slice image” according to the technology of the present disclosure. The slice image SL is reconstructed based on the projection data PD by using, for example, a filtered back projection method.

In the CT apparatus 11, in a case in which the imaging is performed, a plurality of slice images SL are sequentially generated. For example, in a case in which the projection data PD is output from the head side of the subject H as in the present example, the projection data PD is output in order from the head side. Then, the console 17 executes reconstruction processing of the slice images SL in order from the projection data PD on the head side, and sequentially stores the generated slice images SL in the storage 33. The imaging via the CT apparatus 11 ends by generating the slice images SL of the entire imaging range in the body axis direction (Z axis direction). A slice image group including the plurality of slice images SL is referred to as a slice volume VOL. The slice volume VOL is composed of the plurality of slice images SL that are sequentially stored. The slice volume VOL is composed of the slice images SL of a part of the imaging range during the imaging, and is composed of all the slice images SL of the imaging range after the imaging ends.

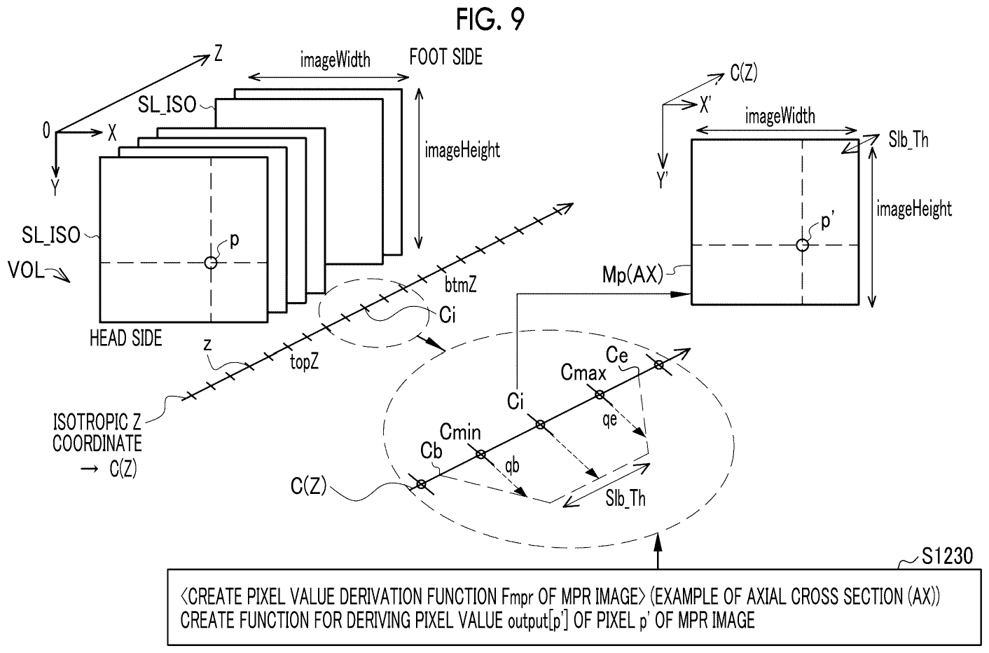

In the MPR image generation processing, first, function creation processing of creating an MPR image generation function GF1 for generating the MPR image is executed. The MPR image generation function GF1 is a function in which the slice image SL is set as an input image and an MPR image Mp is set as an output image. The MPR image generation function GF1 is an example of a function having pixel values of the plurality of slice images SL as parameters. The MPR image Mp is a tomographic image representing a cross section of the subject H that is randomly designated, and is an example of a “tomographic image representing a designated cross section” according to the technology of the present disclosure. In the present example, as the cross section, in addition to an axial cross section (AX), a sagittal cross section (SAG), which is a vertical cross section of the subject H, and a coronal cross section (COR), which is a horizontal cross section of the subject H, will be described as examples (see FIGS. 9 to 11). An X′ axis and a Y′ axis with dashes represent coordinate axes of the cross section of the MPR image Mp. An X′-Y′ plane changes depending on a direction of the designated cross section, and thus may not coincide with the X-Y plane of the slice image SL.

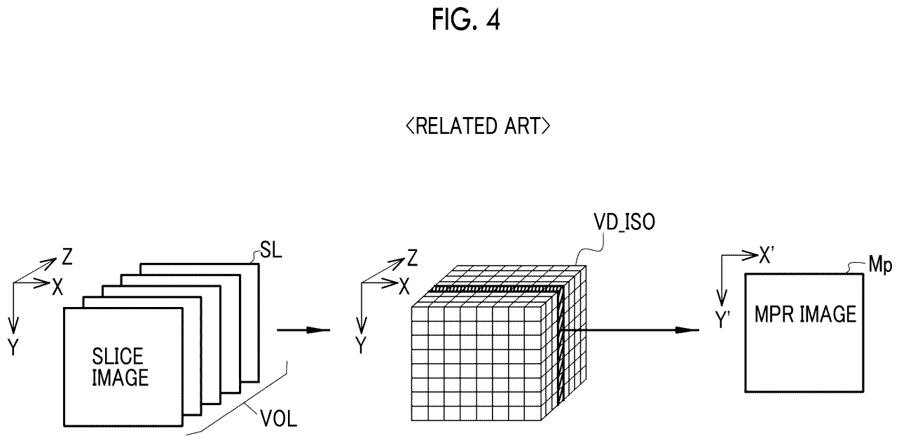

As shown in FIG. 4 as the related art, a method of acquiring the MPR image Mp by designating any cross section in an isotropic three-dimensional image VD_ISO and cutting out the designated any cross section is common. The isotropic three-dimensional image VD_ISO is generated by performing isotropic processing of making the resolutions in the X axis direction, the Y axis direction, and the Z axis direction the same based on the slice volume VOL including all the slice images SL in the imaging range.

As described above, the resolutions of the slice image SL in the X axis direction and the Y axis direction change in accordance with the FOV. For example, the number of pixels of the slice image SL in the X axis direction and the Y axis direction is 512×512. In a case in which the number of pixels is the same, the resolution is lower as the FOV is larger, and the resolution is higher as the FOV is narrower. For example, in a case in which a case in which the FOV is 320 mm and a case in which the FOV is 640 mm are compared, pixel intervals of the slice images SL are 320 mm/512=0.625 and 640 mm/512=1.25, respectively, and the pixel interval is smaller as the FOV is smaller, so that the resolution is higher.

On the other hand, since the slice interval defining the resolution in the Z axis direction is generally longer than the pixel intervals in the X axis direction and the Y axis direction, interpolation processing of the slice image SL is performed to match the pixel interval in the X axis direction and the Y axis direction. This is the isotropic processing. The isotropic three-dimensional image VD_ISO is a three-dimensional image in which the three-dimensional resolution is made isotropic by interpolating the slice images SL based on all the slice images SL of the imaging range, and is composed of isotropic voxels that are pixels made isotropic three-dimensionally. In a case in which the isotropic three-dimensional image VD_ISO is generated, it is possible to generate the MPR image Mp of any cross section, but the isotropic three-dimensional image VD_ISO takes time to be generated and cannot be generated until all the slice images SL in the imaging range are output.

Therefore, the processor 36 according to the technology of the present disclosure directly generates the MPR image Mp from the slice images SL without generating the isotropic three-dimensional image VD_ISO by using the MPR image generation function GF1.

In FIG. 3, the processor 36 acquires the imaging conditions (A) and tomographic image generation conditions (B) in order to create the MPR image generation function GF1. These conditions are set before the imaging is executed in the CT apparatus 11. The processor 36 receives designation of the imaging conditions (A) and the tomographic image generation conditions (B) by, for example, an operation of the operator through the input device 32.

The imaging conditions (A) include A1: slice interval, A2: FOV, and A3: number of pixels (X, Y). The tomographic image generation conditions (B) include B1: direction and position of cross section, and B2: slab thickness. The direction and the position of the cross section are the direction and the position of the cross section of the MPR image Mp to be generated. In the present example, the axial cross section, the sagittal cross section, or the coronal cross section is designated as the direction of the cross section. In addition, in a case of an MPR image Mp(AxAX of the axial cross section, for example, any position in the Z axis direction is designated as the position of the cross section. In a case of an MPR image Mp(SAG) of the sagittal cross section, any position in the Z axis direction and the X axis direction is designated. In a case of an MPR image Mp(COR) of the coronal cross section, any position in the Z axis direction and the Y axis direction is designated.

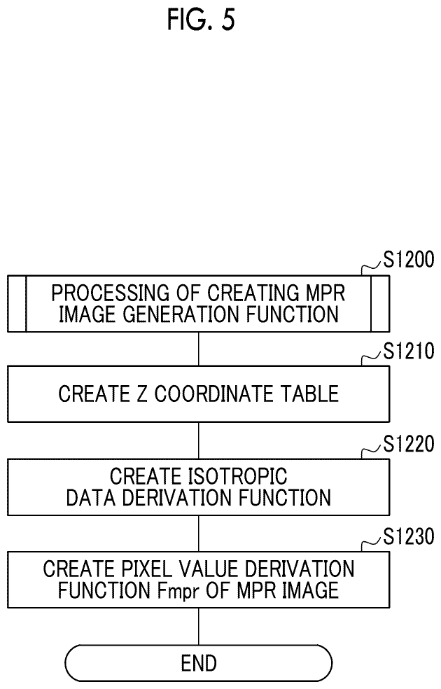

FIG. 5 shows a processing procedure of processing (step S1200) of creating the MPR image generation function GF1. In step S1210, the processor 36 creates a Z coordinate table based on the imaging conditions (A). Then, in step S1220, the processor 36 creates an isotropic data derivation function by using the Z coordinate table, and further, in step S1230, the processor 36 creates a pixel value derivation function Fmpr of the MPR image Mp based on the isotropic data derivation function.

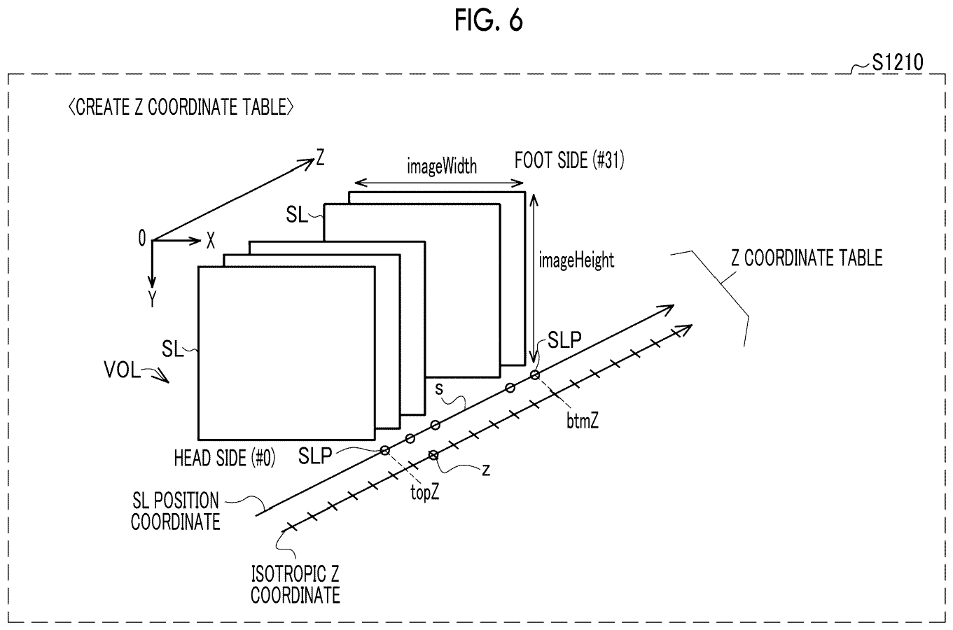

FIG. 6 is a diagram conceptually showing processing of creating the Z coordinate table in step S1210. First, the processing of creating the Z coordinate table of step S1210 will be described with reference to FIG. 6. The Z coordinate table is a table showing a correspondence relationship between an SL position coordinate and the isotropic Z coordinate. The SL position coordinate is a coordinate indicating the positions of the plurality of slice images SL in the Z axis direction, and is derived based on the slice interval included in the imaging conditions (A). In the SL position coordinate, a point s is any position, and a point SLP represents a position at which the slice image SL is present. topZ is a position of the slice image SL located closest to the head side in the reconstructed slice image SL, and btmZ is a position of the slice image SL located closest to the foot side. That is, a range of the slice images SL that can be used for generating the MPR image Mp is shown. In the example of FIG. 6, there are 32 (from #0 to #31) slice images SL in the range from topZ to btmZ.

The isotropic Z coordinate is a Z coordinate with scales set in accordance with the resolutions in the X axis direction and the Y axis direction. That is, the interval between the scales of the isotropic Z coordinate is the same as the pixel interval in the X axis direction and the Y axis direction of the slice image SL. In the isotropic Z coordinate, a point z is any position of the scale.

As shown in FIGS. 7A and 7B, the interval between the scales of the isotropic Z coordinate changes in accordance with the size of the FOV. In a case of comparing a case of a FOV1 shown in FIG. 7A and a case of a FOV2 smaller than the FOV1 shown in FIG. 7B, the interval of the scales is shorter in the FOV2. This is because, as the FOV is smaller, the resolution is higher and the pixel interval in the X axis direction and the Y axis direction is shorter.

In the processing of creating the Z coordinate table in step S1210, the processor 36 first creates the isotropic Z coordinate based on the FOV and the number of pixels (X, Y) set as the imaging conditions (A). Then, a table showing a correspondence relationship between the SL position coordinate and each position z of the isotropic coordinate is created as the Z coordinate table. From the Z coordinate table, in a case in which the position of the MPR image Mp is designated in the isotropic Z coordinate, it is possible to derive the point s of the corresponding SL position coordinate. As will be described later, the designation of the position of the MPR image Mp in the Z axis direction is performed with reference to the scale of the isotropic Z coordinate.

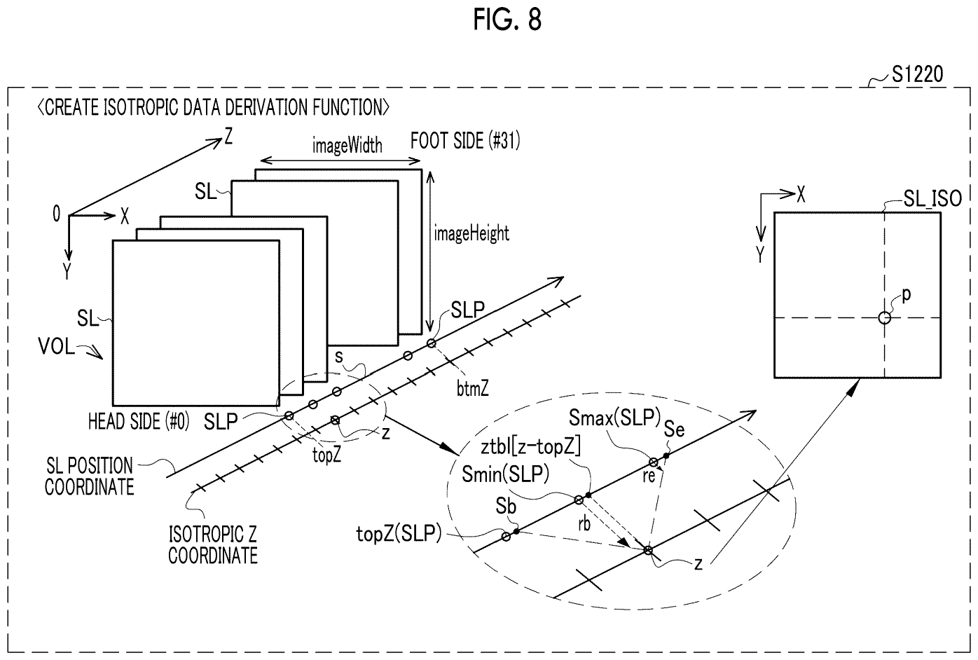

FIG. 8 is a diagram conceptually showing processing of creating the isotropic data derivation function in step S1220. The isotropic data derivation function is a function for deriving an isotropic slice image SL_ISO at each z position of the isotropic Z coordinate. The isotropic slice image SL_ISO is a slice image interpolated at each z position of the isotropic Z coordinate based on the slice image SL. The isotropic data derivation function is a function for deriving a pixel value tmp[z][p] of each pixel p of the isotropic slice image SL_ISO as the isotropic data. Here, [z] indicates a position on the isotropic Z coordinate, and [p] indicates a position in the X-Y plane of the isotropic slice image SL_ISO.

In the processing of creating the isotropic data derivation function in step S1220, the processor 36 first sets the range at the SL position coordinate corresponding to each position z in accordance with the slab thickness of the isotropic slice image SL_ISO and the Z coordinate table. This range is defined by a point Sb on the topZ side and a point Se on the btmZ side, and is a selection range for selecting the slice image SL used for deriving the pixel value tmp[z][p] of the isotropic slice image SL_ISO. The isotropic slice image SL_ISO is a slice image in which a region in which the slice image SL is not present is interpolated. Therefore, the pixel value tmp[z][p] of the isotropic slice image SL_ISO is derived from the pixel values of the plurality of slice images SL present in the vicinity by the linear interpolation. The selection range defines the slice image SL used for the interpolation. A width of the selection range is set in advance in accordance with the interval of the scales of the isotropic Z coordinate. That is, an approximate number of slice images SL used for the interpolation for one z position is set in advance. In the present example, since the number of slice images SL included in the selection range is two (Smin and Smax), two slice images SL are used for the interpolation. It goes without saying that the number of slice images SL used for the interpolation is optional and may be three or more.

In the example shown in FIG. 8, the processor 36 specifies the position of the SL position coordinate corresponding to each position z of the isotropic Z coordinate based on the Z coordinate table. The position corresponding to the z position is set as ztbl[z-topZ]. A point moved by an equal distance to the topZ side and the btmZ side from this position is the point Sb and the point Se, respectively, in the SL position coordinate. The range of the point Sb and the point Se includes Smin(SLP) on the topZ side and Smax (SLP) on the btmZ side. The SLP in parentheses indicates that the slice image SL is present at each of the positions of Smin and Smax. By designating the point Sb and the point Se, two slice images SL to be used for the interpolation are selected.

Then, in the pixel value interpolation processing, the processor 36 obtains how much weight is used for the pixel value of each of the slice images SL at Smin and Smax from an isosceles triangle in which the z position is a vertex and a straight line connecting the point Sb and the point Se is a base. The isosceles triangle represents a weight distribution of the pixel values of the slice images SL to be used. rb is a weighting coefficient to be multiplied by the pixel value of the slice image SL at Smin, and re is a weighting coefficient to be multiplied by the pixel value of the slice image SL at Smax. The maximum value of the weighting coefficient is “1”. In a case of comparing the weighting coefficients of rb and re, rb closer to the z position in the Z axis direction is larger. The processor 36 uses the weighting coefficients rb and re derived in this way to determine the weight of the pixel value of each of the slice images SL at Smin and Smax, and obtains the pixel value tmp[z][p] of the isotropic slice image SL_ISO.

Conceptually, the isotropic data derivation function performs such processing to derive the pixel value tmp[z][p] as the isotropic data. In a case in which the body axis direction is the Z axis direction and two directions defining the cross section orthogonal to the body axis direction are the X axis direction and the Y axis direction, the pixel value tmp[z][p] is a pixel value of a pixel in which the resolution is made isotropic in the X axis direction, the Y axis direction, and the Z axis direction. This is data equivalent to the pixel value of the isotropic voxel in the isotropic three-dimensional image VD_ISO.

In the example shown in FIG. 8, the isotropic data derivation function is represented by Expression (1) under the following conditions as an example. As the conditions, first, the ranges of x, y, and z of the isotropic slice image SL_ISO are as follows.

$$\begin{matrix}0 \leq x < imageWidth \\ 0 \leq y < imageHeight \\ topZ \leq z \leq btmZ\end{matrix}$$

In addition, in Expression (1), [p] indicates a position of the isotropic slice image SL_ISO in the X-Y plane, and is defined by a component [x] in the X axis direction and a component [y] in the Y axis direction.

$$tmp [ z ] [ p ] = rb \times vol [ S min ] [ p ] + {\sum }_{S = Smin + 1}^{Smax - 1} vol [ s ] [ p ] + re \times vol [ S max ] [ p ] \qquad ( 1 )$$

Here, in Expression (1), “vol” indicates a pixel value of the slice image SL included in the slice volume VOL. For example, vol[Smin][p] indicates a pixel value of each pixel p in the X-Y plane of the slice image SL at Smin on the SL position coordinate. vol[Smax][p] indicates a pixel value of each pixel p of the slice image SL at Smax in the X-Y plane on the SL position coordinate. The definitions of the other symbols are as follows.

$$\begin{matrix}rb = S min - Sb \\ re = Se - S max \\ S min = Ceil ( Sb ) \\ S max = floor ( Se ) \\ Sb = ztb 1 [ z - topZ ] - 1 \\ Se = ztb 1 [ z - topZ ] + 1\end{matrix}$$

For example, it is assumed that the position of the SL position coordinate of ztbl[z-topZ] is “2.3”. Then, from each of the expressions of Sb and Se, Sb is “1.3” and Se is “3.3”. Since the slice image SL is used for the interpolation, in order to obtain the position (SLP) of the slice image SL in the vicinity of Sb and Se, “1.3” of Sb is rounded up to obtain an integer value “2”, and Smix is specified. On the other hand, “3” as an integer value is obtained by truncating “3.3” of Se, and Smax is specified. Since the weighting coefficients rb and re of the pixel values of the slice images SL are determined in accordance with the distance from the z position, rb=2−1.3=0.7 and re=3.3−3=0.3. A value obtained by adding 70% of the pixel value of Smin and 30% of the pixel value of Smax is tmp[z][p] at z=2.3. Here, in order to obtain an accurate pixel value, it is necessary to divide the arithmetic mean of the pixel values of the slice images SL at Smin and Smax by the number of used slice images SL. However, as will be described later, tmp[z][p] is data that is integrated into the pixel value derivation function Fmpr of the MPR image Mp. Therefore, tmp[z][p] is used without being divided by the number of slice images SL.

In addition, in the present example, since the slab thickness, which is the thickness of the isotropic slice image SL_ISO in the Z axis direction, is “0”, the slice image SL is not present between Smin and Smax. Therefore, the second term including Σ in Expression (1) is “0”. However, in a case in which the slab thickness is larger than “0” and the slice image SL is present between Smin and Smax, the second term is a value larger than “0”. In a case in which the slab thickness is larger than “0”, the isosceles triangle representing the distribution of the weighting coefficients has a trapezoid shape having an upper base having a length corresponding to the thickness of the slab thickness. The weighting coefficient of the pixel value of the slice image SL defined in the second term is the maximum value of “1”.

FIG. 9 is a diagram conceptually showing processing of creating the pixel value derivation function Fmpr of the MPR image Mp in step S1230. The pixel value derivation function Fmpr shown in FIG. 9 is a function for deriving a pixel value output [p′] of a pixel p′ of the MPR image Mp(AX) of the axial cross section (AX). In the MPR image Mp(AX) of the axial cross section (AX), the X′-Y′ plane represents the cross section, and a C axis represents a thickness direction of a slab thickness Slb_Th. In a case of the MPR image Mp(AX) of the axial cross section (AX), the C axis corresponds to an isotropic Z coordinate. The C axis is denoted as C(Z) to mean that the C axis corresponds to the isotropic Z coordinate. The position of the MPR image Mp(AX) is designated in the isotropic Z coordinate. Ci is a designation position of the cross section of the MPR image Mp(AX), and is designated from the position corresponding to the scale of the isotropic Z coordinate.

A method of deriving the pixel value of the MPR image Mp(AX) is substantially the same as a method of deriving the pixel value of the isotropic slice image SL_ISO shown in FIG. 8. That is, the processor 36 derives the pixel value output [p′] of each pixel p′ of the MPR image Mp(AX) by performing the linear interpolation from the pixel value of the isotropic slice image SL_ISO corresponding to the designation position Ci. In the C(Z) axis, the trapezoid indicated by a dotted line is the same as the isosceles triangle as the distribution of the weighting coefficients of the isotropic data derivation function shown in FIG. 8, and the slab thickness Slb_Th is larger than “0” in FIG. 9, so that the trapezoid is formed. The trapezoid has a width of equal distance on each of the topZ side and the btmZ side with the designation position Ci as a center. In the C(Z) axis, a range defined by Cb and Ce is the selection range of the isotropic slice image SL_ISO used for the interpolation, as in Sb and Se shown in FIG. 8. The slab thickness Slb_Th is designated as the tomographic image generation conditions (B), and the selection range is set in accordance with the designated slab thickness Slb_Th. qb and qe are weighting coefficients to be multiplied by the pixel values of the isotropic slice image SL_ISO at Cmin and Cmax.

The pixel value derivation function Fmpr of the MPR image Mp(AX) conceptually performs such processing to derive the pixel value output [p′].

In the example shown in FIG. 9, the pixel value derivation function Fmpr of the MPR image Mp(AX) is represented by Expression (2) under the following conditions as an example. As the conditions, first, the ranges of x′, y′, and cp of the MPR image Mp(AX) are as follows. cp is a position of the isotropic slice image SL_ISO that is present within the range corresponding to the slab thickness Slb_Th in the isotropic Z coordinate corresponding to the C(Z) axis.

$$\begin{matrix}0 \leq {x}^{′} < imageWidth \\ 0 \leq {y}^{′} imageHeight \\ topZ \leq cp \leq btmZ\end{matrix}$$

In Expression (2), [p′] indicates a position [x′][y′] in the X′-Y′ plane of the MPR image Mp(AX).

$$ouput [ {p}^{′} ] = \frac{qb \times tmp [ C min ] [ {p}^{′} ] + {\sum }_{cp = Cmin + 1}^{Cmax - 1} tmp [ cp ] [ {p}^{′} ] + qe \times tmp [ C max ] [ {p}^{′} ]}{Δ T + 1} \qquad ( 2 )$$

Here, in Expression (2), “tmp” is a pixel value of the isotropic slice image SL_ISO obtained from the isotropic data derivation function. For example, tmp[Cmin][p′] is a pixel value of each pixel p′ in the X-Y plane of the isotropic slice image SL_ISO at Cmin in the isotropic Z coordinate. tmp[Cmax][p′] is a pixel value of each pixel p′ in the X-Y plane of the isotropic slice image SL_ISO at Cmax in the isotropic Z coordinate. The definitions of the other symbols are as follows.

$$\begin{matrix}qb = C min - Cb \\ re = Ce - C max \\ C min = Ceil ( Cb ) \\ C max = floor ( Ce ) \\ Cb = Ci - Δ T / 2 - 1 \\ Ce = Ci + Δ T / 2 + 1 \\ Δ T = S 1 b\_Th / PS\end{matrix}$$

qb and qe correspond to rb and re of the isotropic data derivation function and are weighting coefficients multiplied by the pixel values at Cmin and Cmax. Similarly to rb and re, qb and qe are also obtained in accordance with the weight distribution represented by the trapezoid. AT is a value obtained by dividing the slab thickness Slb_Th in millimeters by a pixel interval PS, and is a thickness corresponding to the slab thickness Slb_Th in units of the scale of the isotropic Z coordinate.

The pixel value derivation function Fmpr includes tmp in Expression (2), and the isotropic data derivation function is integrated. After creating the isotropic data derivation function, the processor 36 creates the pixel value derivation function Fmpr in accordance with the slab thickness Slb_Th designated as the tomographic image generation conditions (B).

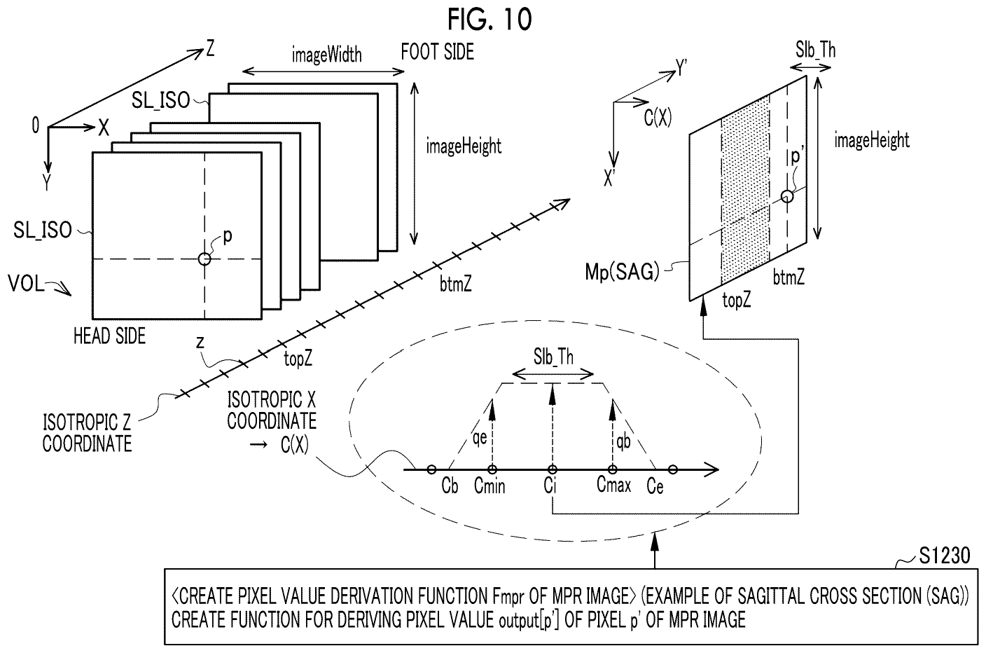

FIG. 10 is a diagram conceptually showing the processing of creating the pixel value derivation function Fmpr for deriving the pixel value output [p′] of the pixel p′ of the MPR image Mp(SAG) of the sagittal cross section (SAG). Basically, the same applies to the example of the axial cross section (AX) shown in FIG. 9. Hereinafter, a difference will be mainly described. In the MPR image Mp(SAG), the X′-Y′ plane represents the cross section, and the C axis represents a thickness direction of the slab thickness Slb_Th. In a case of the MPR image Mp(SAG) of the sagittal cross section (SAG), the C axis corresponds to an isotropic X coordinate. The position of the MPR image Mp(SAG) is designated on the C (X) axis corresponding to the isotropic X coordinate. Ci is a designation position of the cross section of the MPR image Mp(SAG), and is designated from the position corresponding to the scale of the isotropic X coordinate.

Then, the processor 36 obtains the selection range defined by Cb and Ce on the C (X) axis based on the designation position Ci and the slab thickness Slb_Th. The weighting coefficient to be multiplied by the pixel value of the isotropic slice image SL_ISO is obtained from the trapezoidal weight distribution defined by Cb and Ce and the designation position Ci.

In a case of the MPR image Mp(SAG) of the sagittal cross section (SAG), unlike a case of the axial cross section (AX), the isotropic Z coordinate and the direction of the slab thickness Slb_Th are different, and thus the range of the topZ and the btmZ is defined in the width direction (Y′ axis direction) of the X′-Y′ plane.

In the example shown in FIG. 10, the pixel value derivation function Fmpr of the MPR image Mp(SAG) is represented by Expression (3) under the following conditions as an example. As the conditions, first, the ranges of x′, y′, and cp of the MPR image Mp(SAG) are as follows. Here, cp is a pixel position of the isotropic slice image SL_ISO that is present within the range corresponding to the slab thickness Slb_Th at the isotropic X coordinate corresponding to the C (X) axis.

$$\begin{matrix}0 \leq cp < imageWidth \\ 0 \leq {x}^{′} < imageHeight \\ topZ \leq {y}^{′} \leq btmZ\end{matrix}$$

In Expression (3), [p′] indicates a position [x′][y′] in the X′-Y′ plane of the MPR image Mp(SAG).

$$ouput [ {p}^{′} ] = \frac{qb \times tmp [ {y}^{′} ] [ C min ] + {\sum }_{cp = Cmin + 1}^{Cmax - 1} tmp [ {y}^{′} ] [ cp ] + qe \times tmp [ {y}^{′} ] [ C max ]}{Δ T + 1} \qquad ( 3 )$$

In Expression (3), “tmp” as an output value of the isotropic data derivation function is also included, and the isotropic data derivation function is integrated. The definitions of the respective symbols in Expression (3) are as follows, and are the same as those in Expression (2). ΔT is a thickness corresponding to the slab thickness Slb_Th in units of the scale of the isotropic X coordinate.

$$\begin{matrix}qb = C min - Cb \\ re = Ce - C max \\ C min = Ceil ( Cb ) \\ C max = floor ( Ce ) \\ Cb = Ci - Δ T / 2 - 1 \\ Ce = Ci + Δ T / 2 + 1 \\ Δ T = S 1 b\_Th / PS\end{matrix}$$

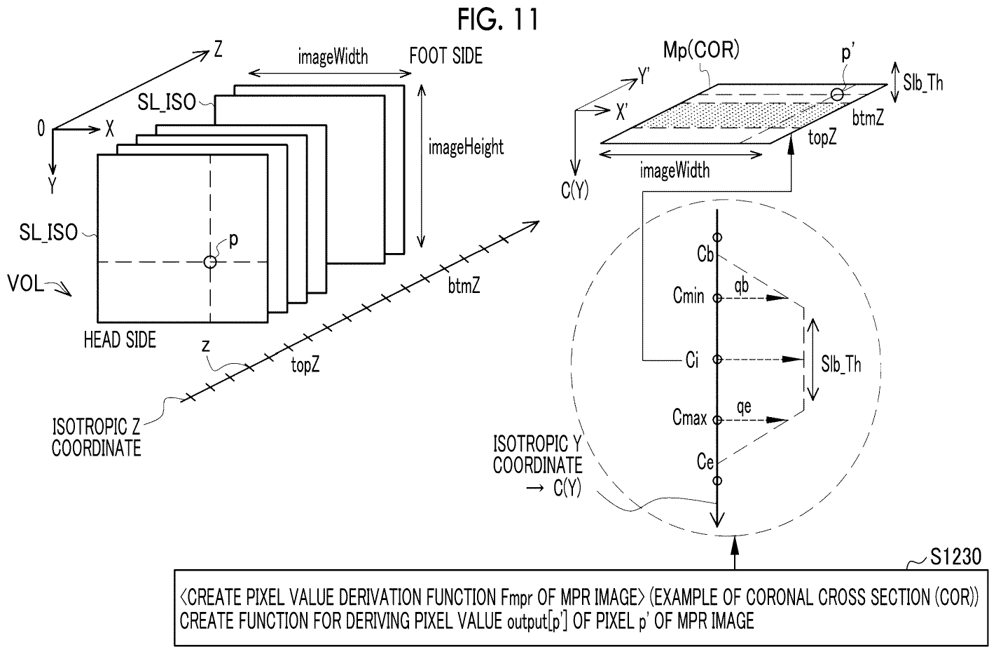

FIG. 11 is a diagram conceptually showing the processing of creating the pixel value derivation function Fmpr for deriving the pixel value output [p′] of the pixel p′ of the MPR image Mp(COR) of the coronal cross section (COR). Basically, the same applies to the example of the sagittal cross section (SAG) shown in FIG. 10. Hereinafter, a difference will be mainly described. In the MPR image Mp(COR), the X′-Y′ plane represents the cross section, and the C axis represents a thickness direction of the slab thickness Slb_Th. In a case of the MPR image Mp(COR) of the coronal cross section (COR), the C axis corresponds to an isotropic Y coordinate. The position of the MPR image Mp(COR) is designated on the C(Y) axis corresponding to the isotropic Y coordinate. Ci is a designation position of the cross section of the MPR image Mp(COR), and is designated from the position corresponding to the scale of the isotropic Y coordinate.

Then, as in a case of the sagittal cross section (SAG), the processor 36 obtains the selection range defined by Cb and Ce on the C(Y) axis based on the designation position Ci and the slab thickness Slb_Th. The weighting coefficient to be multiplied by the pixel value of the isotropic slice image SL_ISO is obtained from the trapezoidal weight distribution defined by Cb and Ce and the designation position Ci.

Even in a case of the MPR image Mp(COR) of the coronal cross section (COR), the range of the topZ and the btmZ is defined in the width direction (Y′ axis direction) of the X′-Y′ plane since the isotropic Z coordinate and the direction of the slab thickness Slb_Th are different as in the sagittal cross section (SAG).

In the example shown in FIG. 11, the pixel value derivation function Fmpr of the MPR image Mp(COR) is represented by Expression (4) under the following conditions as an example. Expression (4) is the same as Expression (3) of the sagittal cross section (SAG). By devising the method of taking the X′ axis and the Y′ axis of the cross section of the MPR image Mp(COR), Expression (3) of the sagittal cross section (SAG) and Expression (4) of the coronal cross section are unified. The conditions are different in a case of the sagittal cross section (SAG). As the conditions, first, the ranges of x′, y′, and cp of the MPR image Mp(COR) are as follows. Here, cp is a pixel position of the isotropic slice image SL_ISO that is present within the range corresponding to the slab thickness Slb_Th at the isotropic Y coordinate corresponding to the C(Y) axis.

$$\begin{matrix}0 \leq {x}^{′} < imageWidth \\ 0 \leq cp < imageHeight \\ topZ \leq {y}^{′} \leq btmZ\end{matrix}$$

In Expression (4), [p′] indicates a position [x′][y′] in the X′-Y′ plane of the MPR image Mp(COR).

$$ouput [ {p}^{′} ] = \frac{qb \times tmp [ {y}^{′} ] [ C min ] + {\sum }_{cp = Cmin + 1}^{Cmax - 1} tmp [ {y}^{′} ] [ cp ] + qe \times tmp [ {y}^{′} ] [ C max ]}{Δ T + 1} \qquad ( 4 )$$

In Expression (4), “tmp” as an output value of the isotropic data derivation function is also included, and the isotropic data derivation function is integrated. The definitions of the respective symbols in Expression (4) are as follows, and are the same as those in Expression (3). ΔT is a thickness corresponding to the slab thickness Slb_Th in units of the scale of the isotropic Y coordinate.

$$\begin{matrix}qb = C min - Cb \\ re = Ce - C max \\ C min = Ceil ( Cb ) \\ C max = floor ( Ce ) \\ Cb = Ci - Δ T / 2 - 1 \\ Ce = Ci + Δ T / 2 + 1 \\ Δ T = S 1 b\_Th / PS\end{matrix}$$

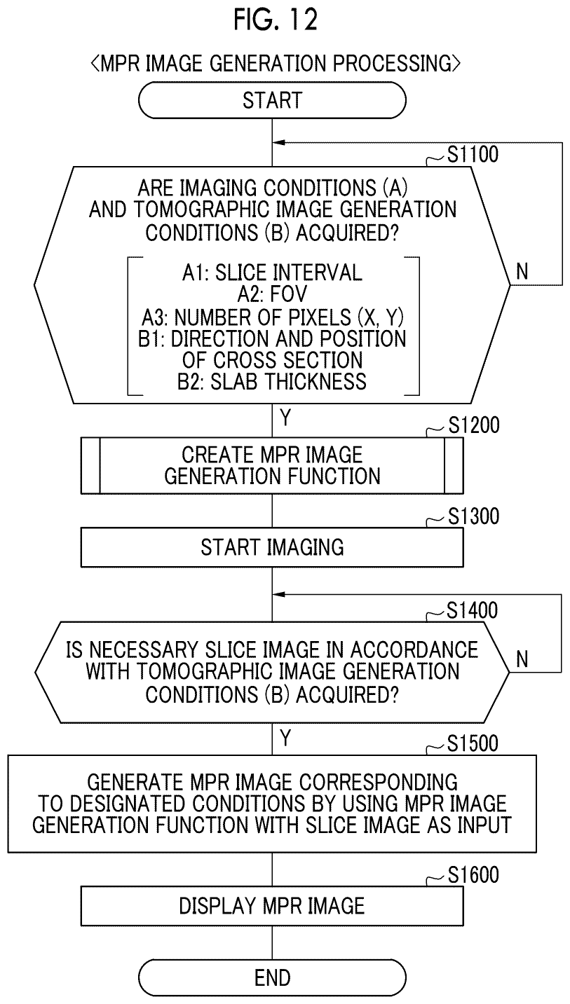

The operation of the above-described configuration will be described with reference to a flowchart showing a processing procedure of the MPR image generation processing shown in FIG. 12. In a case in which the imaging is performed by the CT apparatus 11, in step S1100, the processor 36 of the console 17 waits for the acquisition of the imaging conditions (A) and the tomographic image generation conditions (B). As described in FIG. 2, the imaging conditions (A) include the slice interval, the FOV, and the number of pixels (X, Y). The tomographic image generation conditions (B) include the direction and the position of the cross section of the MPR image Mp and the slab thickness Slb_Th.

In step S1100, in a case in which the imaging conditions (A) and the tomographic image generation conditions (B) are acquired (Y in step S1100), the processor 36 proceeds to step S1200.

In step S1200, the processor 36 acquires the imaging conditions (A) and the tomographic image generation conditions (B) to create the MPR image generation function GF1 before the CT apparatus 11 starts the imaging. The processor 36 creates the MPR image generation function GF1 in the procedure shown in FIG. 5 based on the imaging conditions (A) and the tomographic image generation conditions (B). More specifically, the MPR image generation function GF1 includes the Z coordinate table shown in FIG. 6, the isotropic data derivation function shown in FIG. 8, and the pixel value derivation function Fmpr shown in FIGS. 9 to 11. In step S1200, the processor 36 creates these functions. The imaging conditions (A) of the CT apparatus 11 include at least the slice interval, and the processor 36 creates the MPR image generation function GF1 in accordance with the imaging conditions (A).

Then, in a case in which the imaging is started in step S1300, the processor 36 acquires the projection data PD in order from the head side of the subject H, and starts the generation of the slice image SL based on the acquired projection data PD.

In step S1400, the processor 36 waits for the acquisition of the necessary slice image SL in accordance with the tomographic image generation conditions (B). In a case in which the designation position Ci of the MPR image Mp is on the head side of the subject H, the necessary slice images SL can be acquired relatively quickly. In a case in which the necessary slice images SL are acquired in step S1400, the processor 36 proceeds to step S1500.

In step S1500, the processor 36 generates the MPR image Mp under the designated conditions by using the created MPR image generation function GF1 and inputting the slice image SL. As described above, in a case in which the CT apparatus 11 outputs the plurality of slice images SL during the imaging, the processor 36 starts the processing of generating the MPR image Mp during the imaging.

Then, in step S1600, the processor 36 displays the MPR image Mp on the display 31.

As described above, the console 17 including the processor 36 is an example of an image processing device comprising a processor that performs image processing on the plurality of slice images SL representing the axial cross section (AX) orthogonal to the body axis of the subject H (an example of a subject) output by the CT apparatus 11 (an example of a tomography apparatus). The processor 36 acquires the plurality of slice images SL and generates the MPR image Mp, which is an example of a tomographic image representing a designated cross section, by using the MPR image generation function GF1, which is an example of a function having pixel values of the plurality of slice images SL as parameters.

Therefore, it is possible to acquire the tomographic image, such as the MPR image Mp representing the designated cross section, in a shorter time as compared to the method in the related art in which the isotropic three-dimensional image VD_ISO (see FIG. 4) in which the three-dimensional resolution is made isotropic is generated based on all the slice images SL in the imaging range, and the tomographic image representing the designated cross section is cut out from the generated isotropic three-dimensional image VD_ISO. That is, since a function that receives direct input of the slice image SL and outputs the tomographic image such as the MPR image Mp is used as in the MPR image generation function GF1, the tomographic image representing the designated cross section can be acquired without going through the process of generating the isotropic three-dimensional image. Therefore, it is possible to shorten an acquisition time from the start of the imaging to the acquisition of the tomographic image in the tomography apparatus as compared to the related art.

In addition, as shown in step S1400 and step S1500 of FIG. 12, in a case in which the CT apparatus 11 outputs the plurality of slice images SL during the imaging, the processor 36 according to the technology of the present disclosure starts the processing of generating the tomographic image, such as the MPR image Mp, during the imaging by using the plurality of slice images SL. As described above, since the processing of generating the tomographic image is started during the imaging, the acquisition time of the tomographic image can be further shortened. Here, the term “during the imaging” means from the start of the imaging in the imaging range designated in the body axis direction of the subject to the end of the imaging of the entire imaging range. In the above-described example, for example, in a case in which the entire body of the subject H is the imaging range, the term “during the imaging” means a period from the start of the imaging from the head of the subject H to the end of the imaging of the entire imaging range up to the feet. In addition, as shown in step S1400, the processor 36 can start the generation of the MPR image Mp without waiting for the end of the imaging at a point in time at which the slice image SL necessary for generating the MPR image Mp of the designation position Ci is output, for example.

In addition, the function, such as the MPR image generation function GF1, is created in accordance with the imaging conditions of the CT apparatus 11, the imaging conditions including the slice interval of the slice images SL. Therefore, the processor 36 can create an appropriate function in accordance with the imaging conditions including the slice interval.

In addition, the processor 36 acquires the imaging conditions to create the function, such as the MPR image generation function GF1, before the tomography apparatus starts the imaging. Therefore, it is possible to acquire the tomographic image of the designated cross section, such as the MPR image Mp, in a shorter time as compared to a case in which the imaging conditions are acquired to create the function after the imaging is started. It should be noted that, instead of creating the function for each imaging, for example, a plurality of functions corresponding to different slice intervals may be prepared in advance, and the function may be selected in accordance with the slice interval set as the imaging conditions.

In addition, the MPR image generation function includes an isotropic data derivation function for deriving the isotropic data as the pixel value of the pixel in which the resolution is made isotropic in the X axis direction, the Y axis direction, and the Z axis direction, and the processor generates the tomographic image, such as the MPR image Mp, based on the isotropic data. Therefore, the MPR image Mp equivalent to the MPR image cut out from the isotropic three-dimensional image VD_ISO is obtained. Such an MPR image Mp is useful because a use frequency is high.

In addition, in the above-described example, the cross section of the MPR image Mp, which is an example of the tomographic image, is any one of the axial cross section (AX), the sagittal cross section (SAG), or the coronal cross section (COR). These cross sections are useful because the use frequency is high, and the creation of the MPR image generation function GF1 is simple as compared to a case of generating other cross sections. It should be noted that the cross section of the MPR image Mp may be a cross section in any direction such as an oblique cross section in addition to the cross section of the above-described example. In such a case, a function corresponding to the cross section in any other direction is created.

In addition, the processor 36 receives the designation of the slab thickness as the thickness of the MPR image Mp, which is an example of the tomographic image, and generates the MPR image Mp by performing the weighting in accordance with the slab thickness. Since the weighting is performed in accordance with the slab thickness, the tomographic image in which the information of the subject His accurately reflected is obtained as compared to a case in which the weighting is not performed.

### Second Embodiment

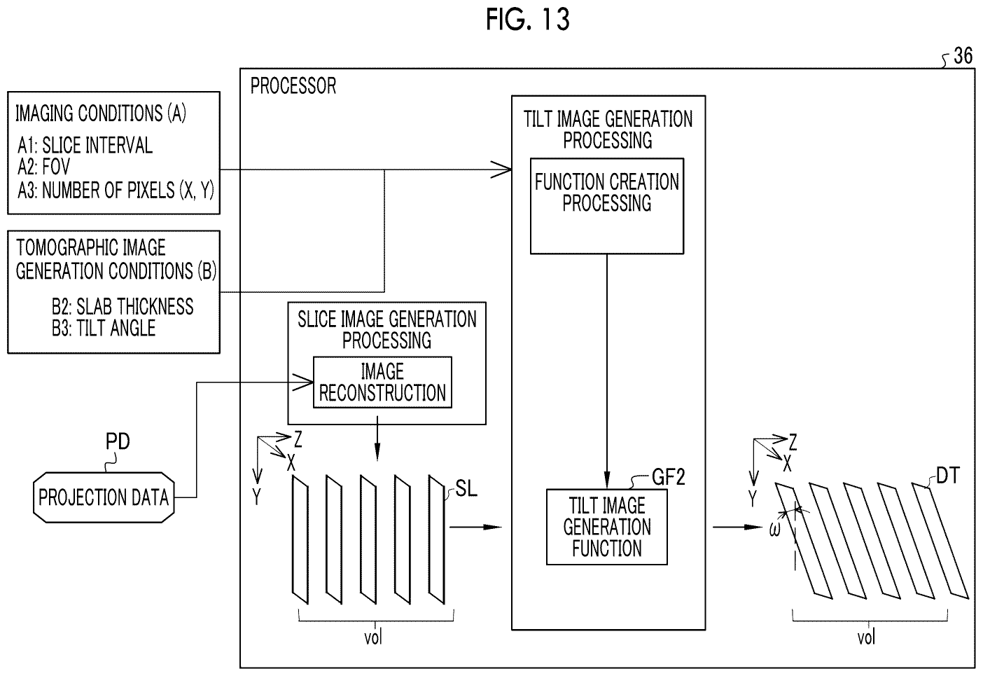

In the second embodiment shown in FIGS. 13 to 19, an example is shown in which the tilt image DT is generated as the tomographic image representing the designated cross section by using a tilt image generation function GF2 having the pixel values of the slice images SL as parameters. The tilt image DT is a tomographic image representing a tilt cross section obtained by tilting the axial cross section of the slice image SL by rotating the axial cross section about the X axis in a case in which the body axis direction is the Z axis direction, the width direction of the cross section orthogonal to the body axis direction is the X axis direction, and the height direction is the Y axis direction.

In the related art, gantry tilt imaging is known in which the gantry 18 is physically tilted to perform the imaging. By tilting the gantry 18, the irradiation direction of the radiation is also tilted with respect to the body axis, and thus the radiation is obliquely transmitted through the subject H. As a result, the slice image SL tilted with respect to the body axis of the subject H is obtained. The slice image SL obtained by the gantry tilt imaging is called a tilt image because the orientation of the cross section is tilted. However, a mechanism for rotating the gantry 18 is complicated and has a large-scale mechanism, so that the cost is very high.

Therefore, in the second embodiment, the tilt image DT is generated by the image processing based on the slice image SL representing the cross section orthogonal to the body axis. The processor 36 creates the tilt image generation function GF2 for generating the tilt image DT based on the imaging conditions (A) and the tomographic image generation conditions (B). The imaging conditions (A) include A1: slice interval, A2: FOV, and A3: number of pixels (X, Y), as in the first embodiment. The tomographic image generation conditions (B) include B2: slab thickness and B3: tilt angle.

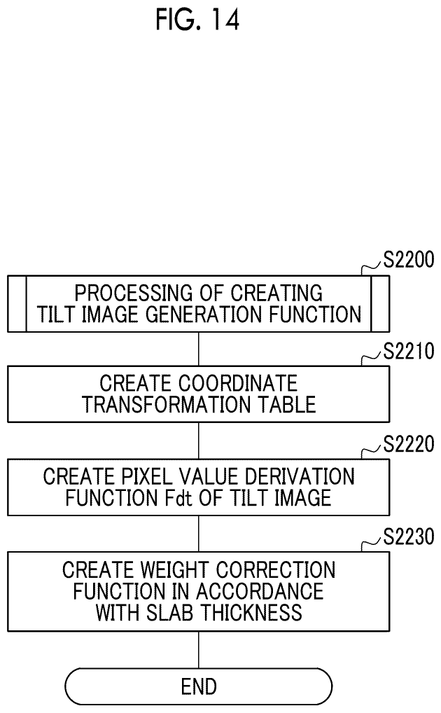

FIG. 14 shows a processing procedure of processing (step S2200) of creating the tilt image generation function GF2. In step S2210, the processor 36 creates a coordinate transformation table. Then, in step S2220, the processor 36 creates a pixel value derivation function Fdt of the tilt image DT, and in step S2230, the pixel value derivation function Fdt of the tilt image DT is created.

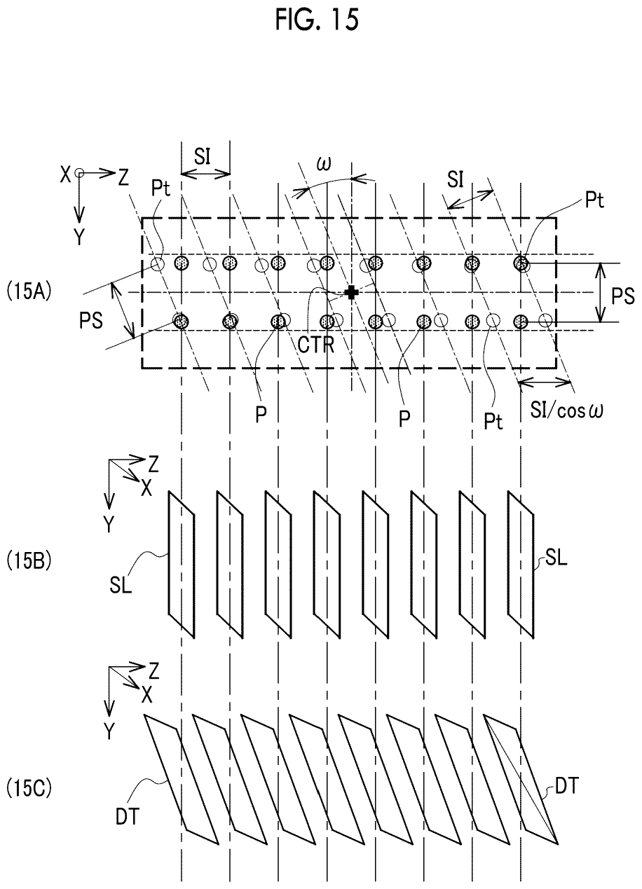

FIG. 15 is a diagram conceptually showing processing of creating the coordinate transformation table in step S2210. As shown in (15A) of FIG. 15, the coordinate transformation table is a table for transforming the position of the pixel P of the slice image SL into the position of the pixel Pt of the tilt image DT. In (15A) of FIG. 15, a thick circle mark with hatching of dots represents the pixel P, and a thin circle mark without hatching represents the pixel Pt.

More specifically, as shown in (15A) of FIG. 15, the coordinate transformation table is a table for performing linear transformation for setting a tilt center CTR and rotationally moving the pixel P about the X axis with the set tilt center CTR as a rotation center. The coordinate transformation table is created based on, for example, a rotation determinant that rotates a point in the Y-Z plane around the X axis with the origin as a center, as shown in Expression (5).

$$[ \begin{matrix}{y}^{′} \\ {z}^{′}\end{matrix} ] = [ \begin{matrix}cos ω & - sin \\ sin ω & cos ω\end{matrix} ] [ \begin{matrix}y \\ z\end{matrix} ] \qquad ( 5 )$$

Here, y and z are coordinates of the pixel P of the slice image SL in the Y-Z plane.

In addition, y′ and z′ are coordinates of the pixel Pt of the tilt image DT in the Y-Z plane.

The processor 36 performs the coordinate transformation using Expression (5) to generate the tilt image DT representing the tilt cross section tilted by rotating the slice image SL of the axial cross section shown in (15B) of FIG. 15 by a tilt angle ω about the X axis as shown in (15C) of FIG. 15.

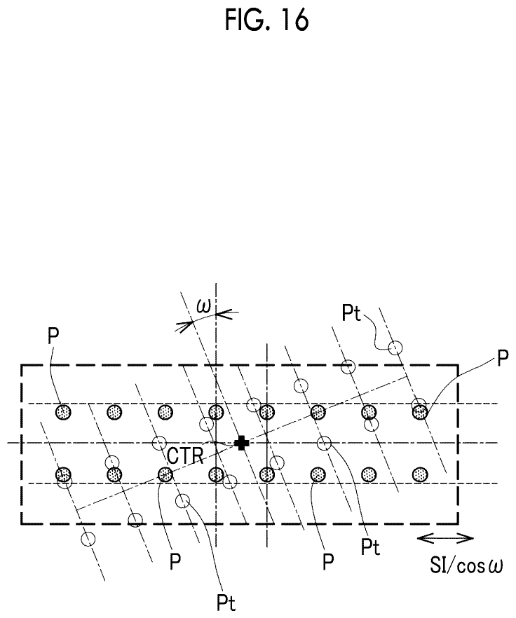

However, as shown in (15A) of FIG. 15, in a plurality of tilt images DT arranged in the Z axis direction, the coordinate transformation is performed such that the heights of the pixels Pt in the Y axis direction are aligned. For example, in a case in which the plurality of slice images SL are simply tilted about the tilt center CTR, as shown in FIG. 16, the pixels P arranged in the Z axis direction in the slice image SL are arranged in the tilt image DT as the pixels Pt arranged in a rightward tilted direction with respect to the Z axis. In addition, in the gantry tilt imaging in which the gantry 18 is tilted, the gantry 18 moves in the Z axis direction in a tilted posture, and the detector 22 has a width. Therefore, the height of the pixel Pt in the Y axis direction changes in a stepwise manner in accordance with the width of the detector 22. As described above, a case in which the height of the pixel Pt changes in the Y axis direction may not be preferable in terms of visibility or the like, and thus, in the tilt image DT according to the technology of the present disclosure, the heights of the pixels Pt in the Y axis direction are aligned. For example, the height of the pixel Pt in the Y axis direction is constant.

As shown in (15A) of FIG. 15, the slice interval SI of the slice images SL and the interval in the directions orthogonal to the two tilted axes tilted in the tilt image DT are the same. The interval between the two tilted axes in the Z axis direction is 1/cos ω times wider than the slice interval SI. In addition, the pixel interval PS between two pixels P adjacent to each other in the Y axis direction of the slice image SL and the pixel interval PS between two pixels Pt adjacent to each other in the tilt axis direction in the tilt image DT are the same. The coordinate transformation table is set to satisfy such conditions. As described in the first embodiment, the pixel interval PS is obtained based on the number of pixels (X, Y) and the FOV set as the imaging conditions (A).

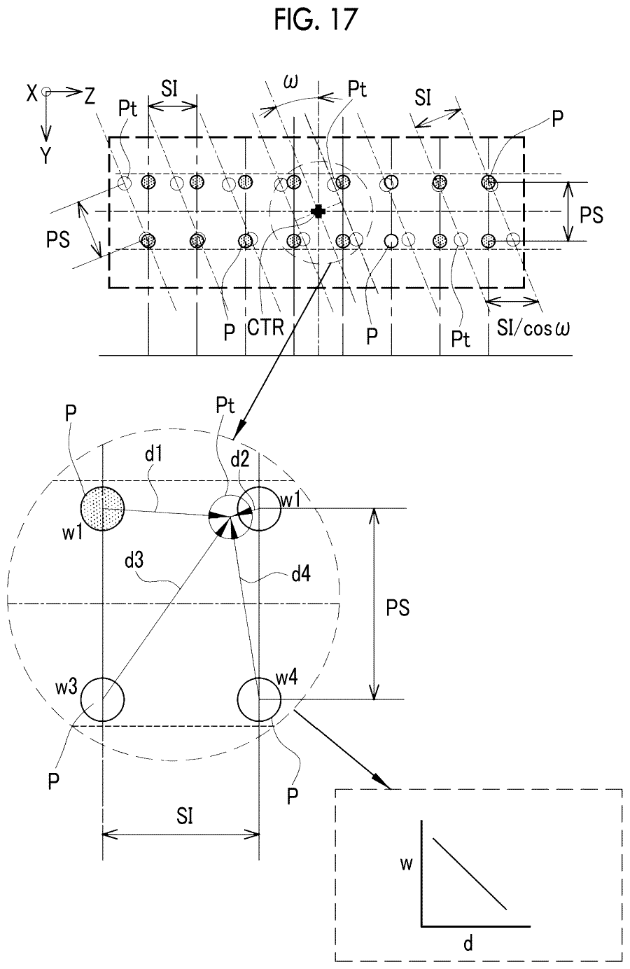

FIG. 17 is a diagram conceptually showing processing of creating the pixel value derivation function Fdt in step S2220. The pixel value of the pixel Pt of the tilt image DT is obtained by the linear interpolation in accordance with the distance d from the pixel value of the pixel P in the vicinity of four points arranged to surround the pixel Pt. In a case in which the distance between the four points of the pixels P and the pixel Pt is d and the weighting coefficient of the pixel values of the four points is w, as in a graph showing a relationship between d and w, the weighting coefficient w is larger as d is smaller. That is, as the distance to the pixel Pt is closer, the weight of the pixel value of the pixel P is greater. The pixel value derivation function Fdt is created in accordance with such conditions.

In a case in which the slab thickness Slb_Th of the tilt image DT is “0”, the generation of the tilt image DT may end only by performing the coordinate transformation and the linear interpolation of the pixel value by using the pixel value derivation function Fdt. However, in a case in which the slab thickness Slb_Th is larger than “0”, it is preferable to generate the tilt image DT by performing weighting in accordance with the slab thickness Slb_Th, for obtaining the tilt image DT that more accurately reflects the information of the subject H.

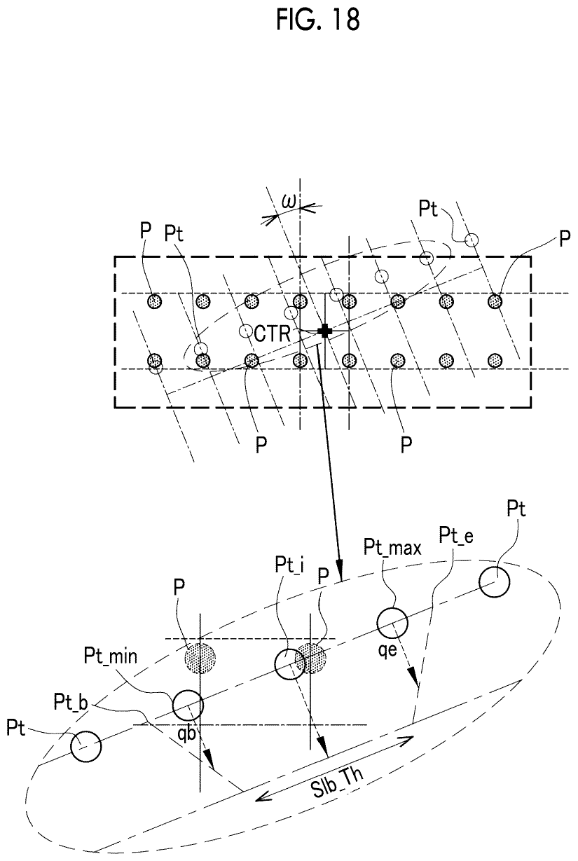

Step S2230 is processing of creating the weight correction function for performing weighting in accordance with the slab thickness Slb_Th. FIG. 18 is a diagram conceptually showing processing of creating the weight correction function in step S2230. In FIG. 18, the pixel Pt_i is the pixel Pt to be subjected to the weight correction in accordance with the slab thickness Slb_Th. The weighting correction method shown in FIG. 18 is the same as the weighting processing shown in FIGS. 9 to 11 according to the first embodiment. That is, the pixel value of the pixel Pt_i to be corrected is corrected by using the pixel values of the plurality of pixels Pt arranged in the thickness direction of the slab thickness Slb_Th and multiplying each pixel value by the weighting coefficient corresponding to the distance from the pixel Pt_i to be corrected. The range defined by Pt_b and Pt_e is the selection range of the pixel Pt used for the correction, and is set in accordance with the slab thickness Slb_Th. The pixel Pt_min and the pixel Pt_max are pixels Pt disposed at both ends within the selection range. qb and qe are weighting coefficients for setting weights of the pixel Pt_min and the pixel Pt_max. In step S2230, the processor 36 creates such a weight correction function based on the slab thickness Slb_Th.

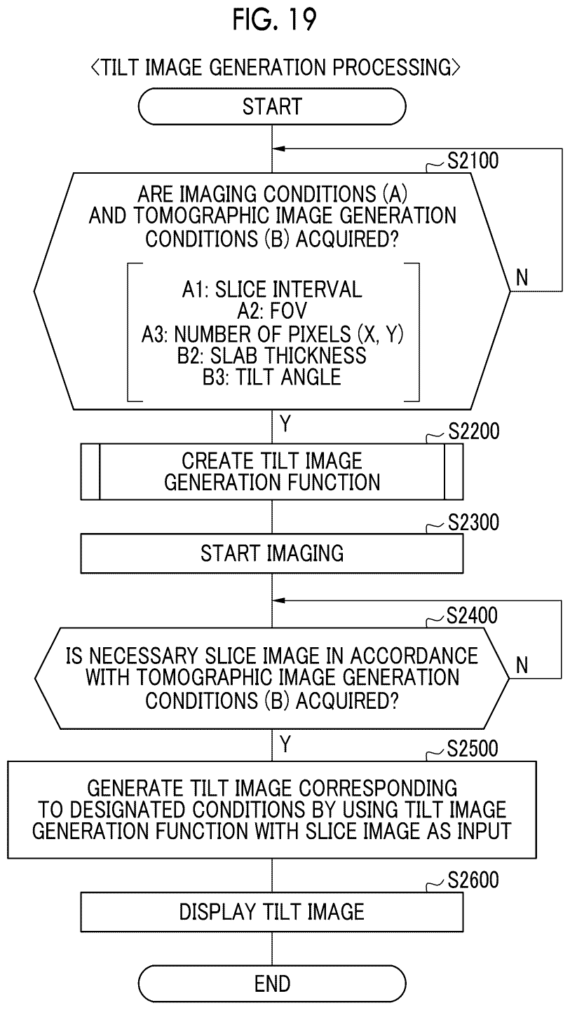

The operation of the above-described configuration will be described with reference to a flowchart showing a processing procedure of the tilt image generation processing shown in FIG. 19. In a case in which the imaging is performed by the CT apparatus 11, in step S2100, the processor 36 of the console 17 waits for the acquisition of the imaging conditions (A) and the tomographic image generation conditions (B). As described in FIG. 13, the imaging conditions (A) include the slice interval, the FOV, and the number of pixels (X, Y). The tomographic image generation conditions (B) include the slab thickness Slb_Th and the tilt angle ω.

In step S2100, in a case in which the imaging conditions (A) and the tomographic image generation conditions (B) are acquired (Y in step S2100), the processor 36 proceeds to step S2200.

In step S2200, the processor 36 creates the tilt image generation function GF2. The processor 36 creates the tilt image generation function GF2 in the procedure shown in FIG. 13 based on the imaging conditions (A) and the tomographic image generation conditions (B). More specifically, the tilt image generation function GF2 includes the coordinate transformation table shown in FIG. 15, the pixel value derivation function Fdt by the four-point interpolation shown in FIG. 17, and the weight correction function shown in FIG. 18. In step S2200, the processor 36 creates these functions. As described above, the processor 36 creates the tilt image generation function GF2 in accordance with the imaging conditions (A) of the CT apparatus 11 including the slice interval SI.

Then, in a case in which the imaging is started in step S2300, the processor 36 acquires the projection data PD in order from the head side of the subject H, and starts the generation of the slice image SL based on the acquired projection data PD.

In step S2400, the processor 36 waits for the acquisition of the necessary slice image SL in accordance with the tomographic image generation conditions (B). In a case in which the designation position of the tilt image DT is on the head side of the subject H, the necessary slice images SL can be acquired relatively quickly. In a case in which the necessary slice image SL is acquired in step S2400, the processor 36 proceeds to step S2500.

In step S2500, the processor 36 uses the created tilt image generation function GF2 and generates the tilt image DT in accordance with the designated conditions such as the tilt angle o with the slice image SL as an input. As described above, in a case in which the CT apparatus 11 outputs the plurality of slice images SL during the imaging, the processor 36 starts the processing of generating the tilt image DT during the imaging.

In step S2600, the processor 36 displays the tilt image DT on the display 31.

As described above, the processor 36 generates the tilt image DT representing the tilt cross section tilted by rotating the axial cross section of the slice image SL about the X axis by using the tilt image generation function GF2 including the pixel value derivation function Fdt. By using the tilt image generation function GF2, the tilt image DT can be obtained at a low cost as compared to a case in which the gantry tilt imaging is performed.

In addition, in a case in which the processor 36 generates the plurality of tilt images DT arranged in the Z axis direction, the heights of the pixels Pt of the plurality of tilt images DT in the Y axis direction are aligned. Therefore, the visibility of the tilt image DT is better as compared to a case in which the heights of the pixels Pt in the Y axis direction are not aligned as shown in FIG. 16 as an example.

The processor 36 receives the designation of the slab thickness Slb_Th as the thickness of the tilt image DT, and generates the tilt image DT by performing weighting in accordance with the designated slab thickness Slb_Th. As described above, by performing the weighting, the tilt image DT in which the information on the subject His accurately reflected is obtained.

It should be noted that, in addition to the tilt image generation processing, the processor 36 may execute the MPR image generation processing shown in the first embodiment.

In addition, as shown in the above-described embodiments, the “function” according to the technology of the present disclosure may be in a form of table data or in a form of an arithmetic expression.

### Third Embodiment

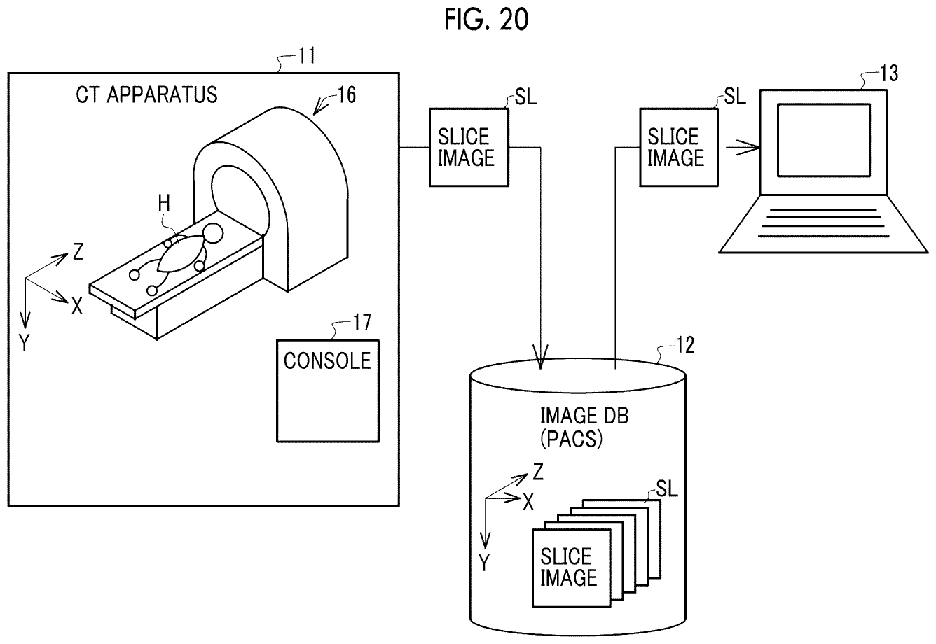

In the above-described embodiments, the console 17 has been described as an example of the image processing device that performs the image processing of the MPR image generation processing and the tilt image generation processing, but as shown in FIG. 20, the image display device 13 different from the console 17 may function as the image processing device. In such a case, for example, the slice image SL output by the CT apparatus 11 is temporarily stored in the image DB 12. The image DB 12 is, for example, a picture archiving and communication system (PACS). The image DB 12 stores the slice image SL of the subject H output from the CT apparatus 11, and distributes the stored slice image SL to the image display device 13 as a request source in response to a request from the image display device 13. The image display device 13 includes the same processor as the processor 36 and executes the image processing, such as the MPR image generation processing and the tilt image generation processing, based on the distributed slice image SL. As an example, the image display device 13 is disposed in each medical department in the medical facility and is used by a doctor in the medical department.

In this case, the tomographic image generation conditions (B) are designated by the doctor of the medical department. In addition, the image display device 13 acquires the imaging conditions (A) set in the CT apparatus 11 from the CT apparatus 11 through the console 17. In the image display device 13, the image processing, such as the MPR image generation processing and the tilt image generation processing, is executed based on the imaging conditions (A) and the tomographic image generation conditions (B).

In the above-described embodiment, the example has been described in which the image processing, such as the MPR image generation processing, is performed based on the slice image SL output during the imaging of the CT apparatus 11, but the image processing, such as the MPR image generation processing, may be performed after the imaging ends. Even in this case, since the process of generating the isotropic three-dimensional image VD_ISO is not necessary, the effect of being able to execute the MPR image generation processing or the tilt image generation processing in a shorter time can be obtained.

In the above-described embodiments, the CT apparatus 11 has been described as an example of the tomography apparatus, but an MRI apparatus may be used. The radiation is not limited to X-rays and may be y-rays.

In addition, in the above-described embodiments, as the hardware structure of the processor 36 of the console 17 or the processor of the image display device 13, various processors shown later can be used. The various processors include, in addition to a CPU that is a general-purpose processor that executes software (program) to function as various processing units, a programmable logic device (PLD) of which a circuit configuration can be changed after manufacturing, such as a field-programmable gate array (FPGA), and a dedicated electric circuit that is a processor having a circuit configuration dedicatedly designed for executing specific processing, such as an application specific integrated circuit (ASIC).

Various types of processing described above may be executed by one of the various processors or may be executed by a combination of two or more processors (for example, a combination of a plurality of FPGAs or a CPU and an FPGA) of the same type or different types. A plurality of processing units may be configured by one processor. As an example in which the plurality of processing units are configured by one processor, there is a form in which a processor that realizes all functions of a system including the plurality of processing units by using one integrated circuit (IC) chip is used, such as a system on a chip (SOC).

In this way, as the hardware structure, the various processing units are configured by using one or more of the various processors described above.

Further, the hardware structure of these various processors is, more specifically, an electric circuit (circuitry) in which circuit elements such as semiconductor elements are combined.

In addition to the operation program of the CT apparatus 11 or the image display device 13, the technology of the present disclosure extends to a computer readable storage medium (USB memory or digital versatile disc (DVD)-read only memory (ROM), or the like) that stores the operation program in a non-transitory manner.

The above-described contents and the above-shown contents are the detailed description of the parts according to the technology of the present disclosure, and are merely examples of the technology of the present disclosure. For example, the description of the configuration, the function, the operation, and the effect are the description of examples of the configuration, the function, the action, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate understanding of the parts according to the technology of the present disclosure, the description of common technical knowledge or the like, which does not particularly require the description for enabling the implementation of the technology of the present disclosure, is omitted in the above-described contents and the above-shown contents.

From the above description, the technologies according to the following supplementary notes can be understood.

### Supplementary Note 1

An image processing device comprising: a processor that performs image processing on a plurality of slice images which are output from a tomography apparatus and which represent an axial cross section orthogonal to a body axis of a subject, in which the processor acquires the plurality of slice images, and generates a tomographic image representing a designated cross section by using a function having pixel values of the plurality of slice images as parameters.

### Supplementary Note 2

The image processing device according to supplementary note 1, in which, in a case in which the tomography apparatus outputs the plurality of slice images during imaging, the processor starts processing of generating the tomographic image during the imaging by using the plurality of slice images.

### Supplementary Note 3

The image processing device according to supplementary note 1 or 2, in which the function is created in accordance with an imaging condition of the tomography apparatus, the imaging condition including at least a slice interval of the slice images.

### Supplementary Note 4

The image processing device according to supplementary note 2 or 3, in which the processor acquires the imaging condition to create the function before the tomography apparatus starts the imaging.

### Supplementary Note 5

The image processing device according to any one of supplementary notes 1 to 4, in which, in a case in which a body axis direction of the subject is a Z axis direction and two directions defining a cross section orthogonal to the body axis direction are an X axis direction and a Y axis direction, the function includes an isotropic data derivation function for deriving isotropic data as a pixel value of a pixel in which a resolution is made isotropic in the X axis direction, the Y axis direction, and the Z axis direction, and the processor generates the tomographic image based on the isotropic data.

### Supplementary Note 6

The image processing device according to supplementary note 5, in which the cross section of the tomographic image is any one of an axial cross section, a sagittal cross section, or a coronal cross section.

### Supplementary Note 7

The image processing device according to supplementary note 5 or 6, in which the processor receives designation of a slab thickness as a thickness of the tomographic image, and generates the tomographic image by performing weighting in accordance with the slab thickness.

### Supplementary Note 8

The image processing device according to any one of supplementary notes 1 to 7, in which, in a case in which a body axis direction of the subject is a Z axis direction, a width direction of a cross section orthogonal to the body axis direction is an X axis direction, and a height direction is a Y axis direction, the function includes a pixel value derivation function for deriving a pixel value of a tilt cross section tilted by rotating the axial cross section of the slice image about an X axis, and the processor generates a tilt image that is the tomographic image of the tilt cross section by using the pixel value derivation function.

### Supplementary Note 9

The image processing device according to supplementary note 8, in which, in a case in which a plurality of the tilt images arranged in the Z axis direction are generated, heights of pixels of the plurality of tilt images in the Y axis direction are aligned.

### Supplementary Note 10

The image processing device according to supplementary note 7 or 8, in which the processor receives designation of a slab thickness as a thickness of the tilt image, and generates the tilt image by performing weighting in accordance with the designated slab thickness.

### Supplementary Note 11

An operation method of an image processing device including a processor that performs image processing on a plurality of slice images which are output from a tomography apparatus and which represent an axial cross section orthogonal to a body axis of a subject, the operation method comprising: via the processor, acquiring the plurality of slice images; and generating a tomographic image representing a designated cross section by using a function having pixel values of the plurality of slice images as parameters.

### Supplementary Note 12

An operation program of an image processing device including a processor that performs image processing on a plurality of slice images which are output from a tomography apparatus and which represent an axial cross section orthogonal to a body axis of a subject, the operation program causing the processor to execute a process comprising: acquiring the plurality of slice images; and generating a tomographic image representing a designated cross section by using a function having pixel values of the plurality of slice images as parameters.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All of the documents, the patent applications, and the technical standards described in the present specification are incorporated into the present specification by reference to the same extent as in a case in which the individual documents, patent applications, and technical standards are specifically and individually stated to be described by reference.

### EXPLANATION OF REFERENCES

What is claimed is:

1. An image processing device comprising:
a processor that performs image processing on a plurality of slice images which are output from a tomography apparatus and which represent an axial cross section orthogonal to a body axis of a subject,
wherein the processor
acquires the plurality of slice images, and
generates a tomographic image representing a designated cross section by using a function having pixel values of the plurality of slice images as parameters.

2. The image processing device according to claim 1,
wherein, in a case in which the tomography apparatus outputs the plurality of slice images during imaging,
the processor starts processing of generating the tomographic image during the imaging by using the plurality of slice images.

3. The image processing device according to claim 1,
wherein the function is created in accordance with an imaging condition of the tomography apparatus, the imaging condition including at least a slice interval of the slice images.

4. The image processing device according to claim 3,
wherein the processor acquires the imaging condition to create the function before the tomography apparatus starts the imaging.

5. The image processing device according to claim 1,
wherein, in a case in which a body axis direction of the subject is a Z axis direction and two directions defining a cross section orthogonal to the body axis direction are an X axis direction and a Y axis direction,
the function includes an isotropic data derivation function for deriving isotropic data as a pixel value of a pixel in which a resolution is made isotropic in the X axis direction, the Y axis direction, and the Z axis direction, and
the processor generates the tomographic image based on the isotropic data.

6. The image processing device according to claim 5,
wherein the cross section of the tomographic image is any one of an axial cross section, a sagittal cross section, or a coronal cross section.

7. The image processing device according to claim 5,
wherein the processor receives designation of a slab thickness as a thickness of the tomographic image, and generates the tomographic image by performing weighting in accordance with the slab thickness.

8. The image processing device according to claim 1,
wherein, in a case in which a body axis direction of the subject is a Z axis direction, a width direction of a cross section orthogonal to the body axis direction is an X axis direction, and a height direction is a Y axis direction,
the function includes a pixel value derivation function for deriving a pixel value of a tilt cross section tilted by rotating the axial cross section of the slice image about an X axis, and
the processor generates a tilt image that is the tomographic image of the tilt cross section by using the pixel value derivation function.

9. The image processing device according to claim 8,
wherein, in a case in which a plurality of the tilt images arranged in the Z axis direction are generated,
heights of pixels of the plurality of tilt images in the Y axis direction are aligned.

10. The image processing device according to claim 8,
wherein the processor receives designation of a slab thickness as a thickness of the tilt image, and generates the tilt image by performing weighting in accordance with the designated slab thickness.

11. An operation method of an image processing device including a processor that performs image processing on a plurality of slice images which are output from a tomography apparatus and which represent an axial cross section orthogonal to a body axis of a subject, the operation method comprising:
via the processor,
acquiring the plurality of slice images; and
generating a tomographic image representing a designated cross section by using a function having pixel values of the plurality of slice images as parameters.

12. A non-transitory computer-readable storage medium storing a operation program of an image processing device including a processor that performs image processing on a plurality of slice images which are output from a tomography apparatus and which represent an axial cross section orthogonal to a body axis of a subject, the operation program causing the processor to execute a process comprising:
acquiring the plurality of slice images; and
generating a tomographic image representing a designated cross section by using a function having pixel values of the plurality of slice images as parameters.
